(19) United States
(12) Patent Application Publication
(10) Pub. No.: US 2026/0258116 A1
(43) Pub. Date: Sep. 3, 2026

(54) ANTI-TOH1 ANTIBODIES AND METHODS OF USE THEREOF

(71) Applicant: The Cleveland Clinic Foundation, Cleveland, OH (US)

(72) Inventor: Jeannette Messer, Cleveland, OH (US)

(21) Appl. No.: 19/489,969

(22) PCT Filed: Jun. 7, 2024
(86) PCT No.: PCT/US2024/032936

§ 371 Date: Dec. 4, 2025

Related U.S. Application Data
(60) Provisional application No. 63/506,769, filed on Jun. 7, 2023.

(51) Int. Cl. C07K 16/1203; A61P 31/04; C07K 16/108; C07K 16/116; C07K 16/20; G01N 33/68

(52) U.S. Cl. CPC C07K 16/1207; A61P 31/04; C07K 16/108; C07K 16/116; C07K 16/20; C07K 16/205; G01N 33/6893; C07K 2317/569; C07K 2317/622; G01N 2333/11; G01N 2333/185; G01N 2333/20; G01N 2333/3156; G01N 2333/44; G01N 2333/445; G01N 2800/065; G01N 2800/26

(57) ABSTRACT

The present disclosure relates to anti-ToH1 antibodies, methods of generating anti-ToH1 antibodies, and methods of use of anti-ToH1 antibodies for diagnosis and treatment of microbial disease and/or chronic inflammatory disease, including microbial infection and microbe-associated chronic inflammatory conditions.

### PRIORITY STATEMENT

This application claims priority to U.S. Provisional Application No. 63/506,769, filed Jun. 7, 2023, the entire contents of which are incorporated herein by reference for all purposes.

### STATEMENT REGARDING FEDERAL FUNDING

This invention was made with government support under DK114713 awarded by the National Institutes of Health. The government has certain rights in the invention.

### SEQUENCE LISTING

The text of the computer readable sequence listing filed herewith, titled “CCF_42114_601_SequenceListing”, created Jun. 7, 2023, having a file size of 56,149 bytes, is hereby incorporated by reference in its entirety.

### FIELD OF THE INVENTION

The present disclosure relates to anti-ToH1 antibodies and methods of use thereof for diagnosis and treatment of microbial disease and/or chronic inflammatory disease in a subject.

### BACKGROUND OF THE INVENTION

The acellular mucosal barrier lies over the colon and normally limits physical and biochemical contact between microbes and the host epithelium. When this first barrier fails, microbes adhere to intestinal epithelial cells (IEC), leading microbial disease. High mobility group box 1 (HMGB1) is a multifunctional protein that is produced by many different human cells in response to innate immune sensing of microbes. HMGB1 deficiency is likely relevant for multiple diseases where microbes adhere to host tissues, including infectious diarrheas, colorectal cancer, and IBD. Accordingly, what is needed are methods of treating or preventing microbial disease and/or chronic inflammatory disease, including in subjects with HMGB1 deficiency.

### SUMMARY OF THE INVENTION

In some aspects, provided herein are methods of treating or preventing microbial disease and/or chronic inflammatory disease in a subject, comprising providing to the subject an antibody that binds to target of HGMB1 (ToH1). The antibody may be any antibody described herein, including the scFv/IgG antibodies and nanobodies provided in the accompanying Examples (e.g. the scFv/IgG antibodies F5, F11, G6, or the nanobodies G2 (VHH-G2) and F7 (VHH-F7). In some embodiments, the microbial disease comprises a microbial infection. For example, the microbial infection can be a bacterial infection, viral infection, fungal infection, and/or protozoal infection. In some embodiments, the microbial infection is a bacterial infection. In some embodiments, the chronic inflammatory disease comprises a microbe-associated chronic inflammatory disease. In some embodiments, the chronic inflammatory disease is inflammatory bowel disease, rheumatoid arthritis, non-alcoholic fatty liver disease, type II diabetes, urinary tract infections, pneumonia, or sepsis. In some embodiments, the chronic inflammatory disease is inflammatory bowel disease.

In some aspects, provided herein are methods of diagnosing microbial disease and/or chronic inflammatory disease in a subject, comprising determining levels of target of HGMB1 (ToH1) in a sample obtained from a subject, and determining that the subject has a microbial disease when the level of ToH1 in the sample are equal to or above a threshold value. In some embodiments, determining levels of ToH1 in the sample comprises contacting a sample obtained from a subject with an antibody that binds to target of HGMB1 (ToH1), and detecting the antibody in the sample. The antibody may be any antibody described herein, including the scFv/IgG antibodies and nanobodies provided in the accompanying Examples (e.g. the scFv/IgG antibodies F5, F11, G6, or the nanobodies G2 (VHH-G2) and F7 (VHH-F7)). In some embodiments, the microbial disease comprises a microbial infection. For example, the microbial infection can be a bacterial infection, viral infection, fungal infection, and/or protozoal infection. In some embodiments, the microbial infection is a bacterial infection. In some embodiments, the chronic inflammatory disease comprises a microbe-associated chronic inflammatory disease. In some embodiments, the chronic inflammatory disease is inflammatory bowel disease, rheumatoid arthritis, non-alcoholic fatty liver disease, type II diabetes, urinary tract infections, pneumonia, or sepsis. In some embodiments, the chronic inflammatory disease is inflammatory bowel disease.

In some aspects, provided herein are methods of generating antibodies that bind to target of HMGB1. In some embodiments, provided herein is a method of generating an antibody that binds to target of HGMB1 (ToH1), comprising sequentially immunizing a host with two or more unique peptides and isolating antibodies generated in response to immunization. In some embodiments, each of the two or more unique peptides has the motif [S/T]xExPx[I/V], wherein each x is a variable amino acid. In some embodiments, the two or more unique peptides comprises three unique peptides. In some embodiments, the two or more unique peptides comprises four unique peptides. In some embodiments, the four unique peptides comprise SxExPxI, SxExPxV, TxExPxI, and TxExPxV.

In some aspects, provided herein are anti-ToH1 antibodies. In some embodiments, provided herein is an antibody that binds to target of HGMB1 (ToH1) comprising a heavy chain variable domain comprising complementary determining regions HCDR1, HCDR2, and HCDR3 and light chain variable domain comprising complementary determining regions LCDR1, LCDR2, and LCDR3.

In some embodiments, HCDR1, HCDR2, and HCDR3 comprise amino acid sequences having at least 80% sequence identity to SEQ ID NO: 11, SEQ ID NO: 12, and SEQ ID NO: 13, respectively, and LCDR1, LCDR2, and LCDR3 comprise amino acid sequences having at least 80% sequence identity to SEQ ID NO: 14, SEQ ID NO: 15, and SEQ ID NO: 16, respectively. In some embodiments, HCDR1, HCDR2, and HCDR3 comprise amino acid sequences having at least 80% sequence identity to SEQ ID NO: 17, SEQ ID NO: 18, and SEQ ID NO: 19, respectively, and LCDR1, LCDR2, and LCDR3 comprise amino acid sequences having at least 80% sequence identity to SEQ ID NO: 20, SEQ ID NO: 21, and SEQ ID NO: 22, respectively. In some embodiments, HCDR1, HCDR2, and HCDR3 comprise amino acid sequences having at least 80% sequence identity to SEQ ID NO: 23, SEQ ID NO: 24, and SEQ ID NO: 25, respectively, and LCDR1, LCDR2, and LCDR3 comprise amino acid sequences having at least 80% sequence identity to SEQ ID NO: 26, SEQ ID NO: 27, and SEQ ID NO: 28, respectively.

In some embodiments, HCDR1, HCDR2, and HCDR3 comprise amino acid sequences having at least 90% sequence identity to SEQ ID NO: 11, SEQ ID NO: 12, and SEQ ID NO: 13, respectively, and LCDR1, LCDR2, and LCDR3 comprise amino acid sequences having at least 90% sequence identity to SEQ ID NO: 14, SEQ ID NO: 15, and SEQ ID NO: 16, respectively. In some embodiments, HCDR1, HCDR2, and HCDR3 comprise amino acid sequences having at least 90% sequence identity to SEQ ID NO: 17, SEQ ID NO: 18, and SEQ ID NO: 19, respectively, and LCDR1, LCDR2, and LCDR3 comprise amino acid sequences having at least 90% sequence identity to SEQ ID NO: 20, SEQ ID NO: 21, and SEQ ID NO: 22, respectively. In some embodiments, HCDR1, HCDR2, and HCDR3 comprise amino acid sequences having at least 90% sequence identity to SEQ ID NO: 23, SEQ ID NO: 24, and SEQ ID NO: 25, respectively, and LCDR1, LCDR2, and LCDR3 comprise amino acid sequences having at least 90% sequence identity to SEQ ID NO: 26, SEQ ID NO: 27, and SEQ ID NO: 28, respectively.

In some embodiments, HCDR1, HCDR2, and HCDR3 comprise SEQ ID NO: 11, SEQ ID NO: 12, and SEQ ID NO: 13, respectively, and LCDR1, LCDR2, and LCDR3 comprise SEQ ID NO: 14, SEQ ID NO: 15, and SEQ ID NO: 16, respectively. In some embodiments, HCDR1, HCDR2, and HCDR3 comprise SEQ ID NO: 17, SEQ ID NO: 18, and SEQ ID NO: 19, respectively, and LCDR1, LCDR2, and LCDR3 comprise SEQ ID NO: 20, SEQ ID NO: 21, and SEQ ID NO: 22, respectively. In some embodiments, HCDR1, HCDR2, and HCDR3 comprise SEQ ID NO: 23, SEQ ID NO: 24, and SEQ ID NO: 25, respectively, and LCDR1, LCDR2, and LCDR3 comprise SEQ ID NO: 26, SEQ ID NO: 27, and SEQ ID NO: 28, respectively.

In some embodiments, the antibody comprises a heavy chain variable domain comprising a sequence having at least 80% sequence identity to SEQ ID NO: 1, SEQ ID NO: 3, or SEQ ID NO: 5; and a light chain variable domain comprising a sequence having at least 80% sequence identity to SEQ ID NO: 2, SEQ ID NO: 4, or SEQ ID NO: 6. In some embodiments, the antibody comprises a heavy chain variable domain comprising a sequence having at least 90% sequence identity to SEQ ID NO: 1, SEQ ID NO: 3, or SEQ ID NO: 5; and a light chain variable domain comprising a sequence having at least 90% sequence identity to SEQ ID NO: 2, SEQ ID NO: 4, or SEQ ID NO: 6. In some embodiments, the antibody comprises a heavy chain variable domain comprising a sequence having at least 95% sequence identity to SEQ ID NO: 1, SEQ ID NO: 3, or SEQ ID NO: 5; and a light chain variable domain comprising a sequence having at least 95% sequence identity to SEQ ID NO: 2, SEQ ID NO: 4, or SEQ ID NO: 6. In some embodiments, the antibody comprises a heavy chain variable domain comprising SEQ ID NO: 1 and a light chain variable domain comprising SEQ ID NO: 2; a heavy chain variable domain comprising SEQ ID NO: 3 and a light chain variable domain comprising SEQ ID NO: 4; or a heavy chain variable domain comprising SEQ ID NO: 5 and a light chain variable domain comprising SEQ ID NO: 6. In some embodiments, the antibody is a single chain variable fragment (scFv). In some embodiments, the antibody is an IgG antibody.

In some embodiments, the antibody is a nanobody. In some embodiments, the antibody is a nanobody having at least 80% identity to SEQ ID NO: 29 or SEQ ID NO: 30. In some embodiments, the antibody is a nanobody having at least 90% identity to SEQ ID NO: 29 or SEQ ID NO: 30. In some embodiments, the antibody is a nanobody having at least 95% identity to SEQ ID NO: 29 or SEQ ID NO: 30. In some embodiments, the antibody is a nanobody having at least 98% identity to SEQ ID NO: 29 or SEQ ID NO: 30. In some embodiments, the antibody is a nanobody comprising the sequence of SEQ ID NO: 29 or SEQ ID NO: 30. In some embodiments, the antibody is a nanobody consisting of the sequence of SEQ ID NO: 29 or SEQ ID NO: 30.

Additional embodiments are described herein.

### DESCRIPTION OF THE FIGURES

FIGS. 1A-1F show that HMGB1 is released into colonic mucus in response to the gut microbiota. FIG. 1A shows immunostaining for HMGB1 (yellow) and bisbenzimide H 33258 (Hoescht, blue) in Carnoy's fixed proximal colon sections from HMGB1WT and HMGB1ΔIEC mice. Arrows indicate the epithelial surface. (n=20). FIG. 1B shows HMGB1 concentration in colonic mucus from HMGB1WT and HMGB1ΔIEC mice measured by ELISA. (n=6) FIG. 1C shows immunoblotting for HMGB1 in colonic mucus from HMGB1WT and HMGB1ΔIEC mice. (n=7) FIG. 1D shows immunostaining for HMGB1 (yellow) and Hoescht (blue) in Carnoy's fixed proximal colon sections from SPF and GF C57BL/6 mice. Arrows indicate the epithelial surface. (n=6) FIG. 1E shows immunoblotting for HMGB1 in mucosal scrapings from SPF and GF C57BL/6 mice. (n=4) FIG. 1F shows HMGB1 concentration in stool from SPF and GF C57BL/6 mice measured by ELISA. (n=6) Data are mean±s.d. Significance determined by Student's two-tailed T-tests. Each datapoint represents one individual mouse. Scale bars, 100 μm. Original magnification 400×.

FIGS. 2A-2H show that HMGB1 prevents bacterial invasion into the inner mucus layer of the colon. FIG. 2A shows fluorescence in situ hybridization (FISH) using the EUB338 probe (purple) and Hoescht (blue) in Carnoy's fixed proximal colon sections from HMGB1WT and HMGB1ΔIEC mice. Arrows indicate the epithelial surface. Dotted lines are placed at the epithelial surface and inner edge of the microbial community. Straight line indicates distance between host tissues and microbial community. FIG. 2B shows distance measured between epithelium and bacterial cells in images represented in FIG. 2A. Each datapoint is an average of 5 measurements for one 4 individual mouse. FIG. 2C shows quantitative PCR for the bacterial 16S rRNA gene in 1 cm of colonic tissue from HMGB1WT and HMGB1ΔIEC mice. (n=6). FIG. 2D shows invasion of green fluorescent protein (GFP) labeled E. coli (SWW33) into mucus isolated from HMGB1WT or HMGB1ΔIEC mice. (n=3; 3 replicates). FIG. 2E shows percentage of the total GFP signal in mucus from HMGB1WT vs. HMGB1ΔIEC mice in images represented in (d) (n=3; 3 replicates). FIG. 2F shows appearance of GFP labeled E. coli (SWW33) exposed to buffer (control) or HMGB1. (n=3; 3 replicates). FIG. 2G shows flow cytometry of aggregates in samples of GFP labeled E. coli (SWW33) exposed to buffer (control) or HMGB1. (n=3; 3 replicates). FIG. 2H shows appearance of Syto9 labeled microbiota from C57BL/6 mice exposed to buffer (control) or HMGB1 labeled with AF647. (n=3; 3 replicates). Data are mean±s.d. Significance determined by Student's two-tailed T-tests. Each datapoint represents one individual mouse. Scale bars, 100 μm. Original magnification 400×.

FIGS. 3A-3N show that HMGB1 binds, inactivates, and regulates expression of the bacterial adhesin FimH through an evolutionarily conserved amino acid sequence. FIG. 3A shows amino acid sequence similarities among the known human HMGB1 target proteins Beclin-1 and Atg5 and bacterial FimH. The putative HMGB1 interaction motif was derived from the amino acid sequence similarities between Beclin-1 and Atg5 and common amino acid replacements. FIG. 3B, 3C show flow cytometry of rHMGB1 binding to E. coli (BW25113) knocked out for FimH (ΔFimH) or ΔFimH E. coli complemented with plasmids encoding wild type FimH (ΔFimHWT; TSETPRV (SEQ ID NO: 33)) or FimH mutated in the conserved residues of the putative motif (ΔFimHMUT; ASATARA). Both the percent E. coli positive for HMGB1 (b) and the amount of HMGB1 protein (mean fluorescence intensity (MFI)) bound to each bacterium (c) were assessed. (n=3; 3 replicates) FIG. D shows label transfer of Sulfo-SBED from HMGB1 to recombinant FimH lectin domain (FimHLD). Recipient proteins were wild type FimHLD (WT; TSETPRV (SEQ ID NO: 33)) or FimHLD mutated in the conserved amino acid residues (mutant; ASATARA) of the putative interaction motif. Transfer was assessed in the absence or presence of mannose. (3 replicates). FIG. 3E shows E. coli colony forming units (CFU) adherent to Caco2 IEC as a percent of input. E. coli were treated with buffer, rHMGB1, or mannose prior to addition to the IEC. (n=6; 3 replicates) FIG. 3F shows percentage of FimHLD protein bound to a mannose coated plate in the presence of increasing amounts of rHMGB1. (n=3; 3 replicates) FIG. 3G shows RBC agglutination by E. coli (SWW33) expressing wild type FimH (FimH:TSETPRV (SEQ ID NO: 33)), knocked out for FimH (FimH: KO), or expressing FimH mutated in the ToH1 sequence (FimH: ASATARA and FimH:AAAAAAA). Numbers of bacteria decrease from left to right (3 replicates). FIG. 3H shows immunoblotting for FimH in mucus isolated from HMGB1WT and HMGB1ΔIEC mice (n=4). FIG. 3I shows quantification of band densitometry of immunoblots represented in (h) (n=4). FIG. 3J shows immunostaining for FimH in Carnoy's fixed proximal 7 colon sections from HMGB1WT and HMGB1ΔIEC mice. FIG. 3K shows quantification of FimH positive bacteria in images represented in (j). FIG. 3L shows immunoblotting for FimH in E. coli (SWW33) exposed to increasing amounts of HMGB1 (3 replicates) FIG. 3M shows PCR determination of the orientation of the DNA switch region governing Fim gene expression in E. coli (ΔFimE) treated with media conditioned by IEC organoids derived from HMGB1ΔIEC mice (ΔIEC CM), HMGB1WT mice (WT CM), or ΔIEC CM supplemented with rHMGB1. Phase-on denotes switch oriented toward production of Fim genes. ftsZ is used for normalization. FIG. 3N shows relative band density of phase-on in (1). Data are mean±s.d. Significance determined by Student's two-tailed T-tests for pairwise comparisons or two-way ANOVA with multiple comparisons. Each datapoint represents one individual mouse.

FIGS. 4A-4H show that HMGB1 mucosal defense is compromised in ulcerative colitis. FIG. 4A shows immunostaining for HMGB1 in Carnoy's fixed sections of resected colon from Non-IBD or UC patients (n=16). FIG. 4B shows quantification of surface associated HMGB1 using images represented in (a). Staining intensity reported as relative fluorescent units (RFU) per μm2 (n=16). FIG. 4C shows surface associated HMGB1 reported in (b) graphed by inflammation severity. FIG. 4D shows immunostaining for FimH in Carnoy's fixed sections of resected colon from Non-IBD or UC patients. Serial tissue sections from the same patients represented in (a) (n=16) FIG. 4E shows quantification of FimH positive bacteria using images represented in (d). Reported as number of objects per high powered field. (n=16) FIG. 4F shows quantification of FimH positive bacteria reported in (e) graphed by inflammation severity. FIG. 4G shows surface HMGB1 and FimH positive bacteria plotted for each patient. The size of the closed circles corresponds to inflammation severity. Open ovals denote the population characteristics by group (non-IBD and UC). FIG. 4H shows two-way scatter plot of surface HMGB1 and FimH positive bacteria in each patient with a fitted curve. The relationship between HMGB1 and FimH was captured by non-linear regression. Data are mean±s.d. Each datapoint represents one individual person. Mann-Whitney U tests were used to compare HMGB1 and FimH in non-IBD vs. UC groups (two-group comparison), and Kruskal Wallis tests were used to assess the difference in HMGB1 and FimH among inflammation groups (three-group comparison).

FIGS. 5A-5E shows that HMGB1 interacts with gut microbes, but does not exert strong selection pressure on the overall microbial community. FIG. 5A shows immunostaining for HMGB1 in Carnoy's fixed proximal colon sections from HMGB1WT mice. Focal plane optimized to capture gut microbes. Scale bars, 100 μm. Original magnification 400×. Arrows indicate the leading edge of gut microbes. FIG. 5B shows shannon alpha-diversity index of ASV abundances using DNA isolated from mucosal scrapings of colons from HMGB1WT and HMGB1ΔIEC mice. FIG. 5C shows canonical correspondence analysis (CCA) on ASV abundances using DNA isolated from mucosal scrapings of colons from HMGB1WT and HMGB1ΔIEC mice. FIG. 5D shows dimensional reduction plots used to characterize microbiome differences between the indicated sites (stool and mucosal) in samples from HMGB1WT and HMGB1ΔIEC mice. R2 derived from permutational multivariate analysis of variance with site as the main variable. R2 indicates the difference between the composition of the microbiota at the two sites in mice of each genotype (HMGB1WT and HMGB1ΔIEC) and p-value indicates the significance of the difference in composition between the two sites. FIG. 5E shows mean proportion of statistically different bacterial strains using DNA isolated from mucosal scrapings of colons from HMGB1WT and HMGB1ΔIEC mice. Each datapoint represents one individual mouse except in (d) where each mouse has one datapoint for stool and one for mucosal sample.

FIGS. 6A-6D show HMGB1 binds to E. coli producing FimH containing the ToH1 sequence. FIG. 6A shows immunofluorescence staining of HMGB1 (red) bound to E. coli (BW25113) (green). Wild type E. coli (WT), E. coli knocked out for FimH (ΔFimH), or ΔFimH E. coli complemented with plasmids carrying either WT FimH (ΔFimHWT) or FimH mutated in ToH1 (ΔFimHMut) exposed to rHMGB1 and SYTO 9 to label bacterial DNA. FIG. 6B shows flow cytometry for FimH expression on the surface of ΔFimH E. coli complemented with plasmids carrying either WT FimH (ΔFimHWT) or FimH mutated in ToH1 (ΔFimHMut). FIG. 6C shows flow cytometry gating strategy for HMGB1 biding reported in FIG. 3b, c. FIG. 6 D shows flow cytometry of rHMGB1 binding to E. coli of the indicated strains. e, Flow cytometry gating strategy for HMGB1 binding reported in (d). Data are mean±s.d. Significance determined by Student's two-tailed T-tests. Each datapoint represents one biological replicate.

FIG. 7 shows a bio informative prediction of the HMGB1 target sequence based upon experimental data using a position specific scoring matrix (http://slim.icr.ac.uk/pssmsearch/).

FIG. 8 is a graph showing that the exemplary anti-ToH1 antibody F11 inhibits FimH binding to target ligand mannose.

FIG. 9 shows anti-ToH1 staining of colonic tissue from an HMGB1 deficient mouse using the exemplary antibody F11 (red). Results demonstrate that the antibody is suitable for immunostaining to reveal ToH1 positive microbes in close proximity or attached to the intestinal surface. Blue is host cell DNA and green is E-cadherin, which stains IEC borders.

FIGS. 10A-10B show binding to ToH1 peptides for antibody G6 (FIG. 10A) and F5 (FIG. 10B), verified by ELISA.

FIG. 11 shows ToH1 peptide binding results for antibody VHH-G2 verified by ELISA. VHH-G2 is shown to bind all ToH1 peptides.

FIG. 12 shows VHH-G2 binding to ToH1 positive adhesins by ELISA. Adhesins are Pilin (Streptococcus pneumoniae), Duffy receptor (Plasmodium vivax), Hemagglutinin (Influenza B), Basic membrane protein B (BmpB, Borrelia burgdorferi), Non-structural protein 1 (NSP-1, Dengue virus), and Variant-specific surface protein VSP4A1 (CRISP-90) (VSP4A1, Giardia intestinalis (Giardia lamblia).

FIG. 13 shows VHH-G2 binding to E. coli FimH by ELISA.

FIG. 14 shows VHH-G2 binding to and aggregating E. coli. Bacteria are labeled in green. Scale bar is 50 μm.

FIG. 15 shows VHH-G2 increases bacterial clearance by macrophages. Top images show phase contrast of RAW 267 macrophages and E. coli. Scale bar 20 μm. The bottom bar graph shows percent change in bacterial concentration in the media from cells depicted in the images by colony forming units.

FIG. 16 shows VHH-G2 binding to human IL1R1 by ELISA.

FIG. 17 show binding to ToH1 peptides for antibody VHH-F7.

FIG. 18 shows VHH-F7 binding to ToH1 positive adhesins by ELISA. Adhesins are Pilin (Streptococcus pneumoniae), Duffy receptor (Plasmodium vivax), Hemagglutinin (Influenza B), Basic membrane protein B (BmpB, Borrelia burgdorferi), Non-structural protein 1 (NSP-1, Dengue virus), and Variant-specific surface protein VSP4A1 (CRISP-90) (VSP4A1, Giardia intestinalis (Giardia lamblia).

FIG. 19 shows VHH-F7 binding to E. coli FimH by ELISA.

FIG. 20A shows VHH-F7 binding to and aggregating E. coli. Bacteria are labeled in green. Scale bar is 50 μm. FIG. 20B shows VHH-F7 binding to and aggregating S. aureus. Bacteria are labeled in green. Scale bar is 50 μm. FIG. 20C shows VHH-F7 binding to and aggregating bacteria from a complex community. Bacteria (labeled in green) were isolated from the colon of a B6 mouse and treated with VHH-F7 in vitro. Scale bar is 50 μm. The bacterial community used in this and subsequent assays is composed of hundreds of bacterial species, with confirmed members including Lachnospirachiae, Listeria, Escherichia-Shigella, Bifidobacterium, Lactobacillus, Streptococcus and Staphylococcus (confirmed by 16S rRNA DNA sequencing).

FIG. 21 shows VHH-F7 increases bacterial clearance by macrophages. Top images show phase contrast of RAW 267 macrophages and E. coli. Scale bar 20 μm. The bottom bar graph shows percent change in bacterial concentration in the media from cells depicted in the images by colony forming units.

FIGS. 22A-22C show results from adhesion inhibition assays demonstrating that VHH-F7 inhibits E. coli binding to mannose (FIG. 22A), S. aureus binding to fibronectin (FIG. 22B), and bacterial community binding to fibronectin (FIG. 22C).

FIG. 23A shows VHH-F7 bind to IL1R1 (a mammalian ToH1 positive protein) by ELISA. FIG. 23 shows that VHH-F7 inhibits IL1R signaling.

### DEFINITIONS

To facilitate an understanding of the present invention, a number of terms and phrases are defined below.

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, the term “antibody” is used in the broadest sense and includes antibodies, antibody derivatives, and antibody fragments. Exemplary include, for example, monoclonal antibodies, monospecific antibodies, multispecific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies (e.g. nanobodies), Fab fragments, F(ab′) fragments, F(ab′) 2 fragments, Fc fragments (e.g. the Fc region of an antibody), disulfide-linked Fvs (“sdFv”), and anti-idiotypic (“anti-Id”) antibodies, dual-domain antibodies, dual variable domain (DVD) or triple variable domain (TVD) antibodies, and functionally active epitope-binding fragments of any of the above. In particular, antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2), or subclass.

An antibody fragment, which is included in the term “antibody”, refers to a portion of an intact antibody comprising the antigen-binding site or variable region. In some embodiments, an antibody fragment does not include the constant heavy chain domains (i.e., CH2, CH3, or CH4, depending on the antibody isotype) of the Fc region of the intact antibody. Examples of antibody fragments include, but are not limited to, Fc fragments, Fab fragments, Fab′ fragments, Fab′-SH fragments, F(ab′)₂ fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

An antibody “derivative”, which is included in the term “antibody” as used herein, refers to an antibody having one or more modifications to its amino acid sequence when compared to a parent antibody. An antibody derivative may exhibit a modified domain structure. The derivative may still be able to adopt the typical domain configuration found in native antibodies, as well as an amino acid sequence, which is able to bind to targets (antigens) with specificity. Typical examples of antibody derivatives are antibodies coupled to other polypeptides, rearranged antibody domains, or fragments of antibodies. The derivative may also comprise at least one further compound linked by covalent or non-covalent bonds.

“CDR” is used herein to refer to the “complementarity determining region” within an antibody variable sequence. There are three CDRs in each of the variable regions of the heavy chain and the light chain. Proceeding from the N-terminus of a heavy or light chain, these regions are denoted “CDR1”, “CDR2”, and “CDR3”, for each of the variable regions. The term “CDR set” as used herein refers to a group of three CDRs that occur in a single variable region that binds the antigen. An antigen-binding site, therefore, may include six CDRs, comprising the CDR set from each of a heavy and a light chain variable region. A polypeptide comprising a single CDR, (e.g., a CDR1, CDR2, or CDR3) may be referred to as a “molecular recognition unit.” Crystallographic analyses of antigen-antibody complexes have demonstrated that the amino acid residues of CDRs form extensive contact with bound antigen, wherein the most extensive antigen contact is with the heavy chain CDR3. Thus, the molecular recognition units may be primarily responsible for the specificity of an antigen-binding site. In general, the CDR residues are directly and most substantially involved in influencing antigen binding/

As used herein, the term “co-administration” refers to the administration of at least two agent(s) (e.g., a protein or peptide of the present invention) or therapies to a subject. In some embodiments, the co-administration of two or more agents/therapies is concurrent. In some embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

The term “diagnosed,” as used herein, refers to the recognition of a disease by its signs and symptoms (e.g., resistance to conventional therapies), or genetic analysis, pathological analysis, histological analysis, diagnostic assay (e.g., for disease) and the like.

As used herein, the term “effective amount” refers to the amount of a therapeutic agent (e.g., a protein or peptide of the present invention) sufficient to effect beneficial or desired results. An effective amount can be administered in one or more administrations, applications or dosages and is not intended to be limited to a particular formulation or administration route.

As used herein, the term “host cell” refers to any eukaryotic cell (e.g., mammalian cells, avian cells, amphibian cells, plant cells, fish cells, and insect cells), whether located in vitro or in vivo.

As used herein the term, “in vitro” refers to an artificial environment and to processes or reactions that occur within an artificial environment. In vitro environments include, but are not limited to, test tubes and cell cultures. The term “in vivo” refers to the natural environment (e.g., an animal or a cell) and to processes or reaction that occur within a natural environment.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, PA [1975]).

The term “sample” as used herein is used in its broadest sense. A sample may comprise a cell, tissue, or fluids, fecal or stool samples, nucleic acids or polypeptides isolated from a cell, and the like.

As used herein, the term “subject” refers to organisms to be treated by the methods of embodiments of the present invention. In some embodiments, the subject is a vertebrate. In some embodiments, the subject is a mammal (e.g., murines, simians, equines, bovines, porcines, canines, felines, and the like). In some embodiments, the subject is a human. In some embodiments, the subject is a bird (e.g. chicken). In some embodiments, the subject is a reptile. In the context of the invention, the term “subject” generally refers to an individual who will receive or who has received treatment (e.g., administration of a protein or peptide of the present invention and optionally one or more other agents) for disease (e.g., inflammatory disease) or other condition requiring treatment.

As used herein, the term “toxic” refers to any detrimental or harmful effects on a cell or tissue as compared to the same cell or tissue prior to the administration of the toxicant.

As used herein, the terms “treat,” “treatment,” and “treating” refer to reducing the amount or severity of a particular condition, disease state (e.g. microbial disease) or symptoms thereof, in a subject presently experiencing or afflicted with the condition or disease state. The terms do not necessarily indicate complete treatment (e.g., total elimination of the condition, disease, or symptoms thereof). “Treatment,” encompasses any administration or application of a therapeutic or technique for a disease (e.g., in a mammal, including a human), and includes inhibiting the disease, arresting its development, relieving the disease, causing regression, or restoring or repairing a lost, missing, or defective function; or stimulating an inefficient process.

As used herein, the term “virulence” as in “microbial virulence” refers to the degree of damage (e.g., level of disease) caused by a microbe (e.g., bacteria) to its host (e.g., subject). In some embodiments, virulence of a microbe is related to its intrinsic virulence factors. The virulence factors of bacteria are typically, for example, proteins or other molecules that enable bacteria to cause disease. For example, virulence factors can be adhesion proteins or toxins.

### DETAILED DESCRIPTION OF THE INVENTION

Adhesion is the first step of microbial disease and blocking adhesion has the potential to prevent infection. High mobility group box 1 (HMGB1) is a multifunctional protein that is produced in IEC and other human cells in response to innate immune sensing of microbes. Experiments herein demonstrate that HMGB1 binds to a specific amino acid motif, target of HGMB1 (ToH1), found in a number of bacterial, fungal, viral, and protozoal proteins. Many of these proteins are expressed on the surface of microbes and are associated with microbial virulence and human and animal disease pathophysiology. Herein it is demonstrated that HMGB1 binding to ToH1 prevents microbial adherence proteins from binding to their carbohydrate target on mammalian host cells to anchor the bacteria in place. Failure of this defense is likely relevant for multiple diseases where microbes adhere to host tissues, including infectious diarrheas, colorectal cancer, and IBD. Accordingly, ToH1 provides a novel molecular target for diagnosis and treatment of disease. Provided herein are anti-ToH1 antibodies, methods of generating anti-ToH1 antibodies, and methods of diagnosing and treating microbial disease and/or chronic inflammatory disease using the same.

### I. Anti-ToH1 Antibodies

In some aspects, provided herein are antibodies that bind to ToH1, also referred to herein as anti-ToH1 antibodies. In some embodiments, provided herein is an antibody that binds to target of HGMB1 (ToH1), the antibody comprising a heavy chain variable domain (VH) and a light chain variable domain (VL). In some embodiments, the antibody comprises a VH comprising 3 CDRS, HCDR1, HCDR2, and HCDR3, and a VL comprising 3 CDRS, LCDR1, LCDR2, and LCDR3. In some embodiments, HCDR1, HCDR2, and HCDR3 comprise amino acid sequences having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identity, or 100% identity) to GFIFSNYG (SEQ ID NO: 11), ISGYNGQT (SEQ ID NO: 12), and ARQSIPYYMDV (SEQ ID NO: 13), respectively, and LCDR1, LCDR2, and LCDR3 comprise amino acid sequences having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identity, or 100% identity) to QSLVHSNGNTY (SEQ ID NO: 14), RISNRLSGVPDRES (SEQ ID NO: 15), and MQAKQFPVT (SEQ ID NO: 16), respectively.

In some embodiments, HCDR1, HCDR2, and HCDR3 comprise amino acid sequences having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identity, or 100% identity) to GYTFTGYY (SEQ ID NO: 17), INPNSGGT (SEQ ID NO: 18), and ARDRGSGATRYGMDV (SEQ ID NO: 19), respectively, and LCDR1, LCDR2, and LCDR3 comprise amino acid sequences having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identity, or 100% identity) to SSDIGNYNY (SEQ ID NO: 20), DVTKRPSGVSNRLSGSKSGNT (SEQ ID NO: 21), and SSYTGRSSWV (SEQ ID NO: 22), respectively.

In some embodiments, HCDR1, HCDR2, and HCDR3 comprise amino acid sequences having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identity, or 100% identity) to GDSVSSNSAA (SEQ ID NO: 23), TYYRSKWYN (SEQ ID NO: 24), and ARRSTWGTFDY (SEQ ID NO: 25), respectively, and LCDR1, LCDR2, and LCDR3 comprise amino acid sequences having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identity, or 100% identity) to QSVLYSSNNKNY (SEQ ID NO: 26), WASTRESGVPDRES (SEQ ID NO: 27), and QQYYALPLT (SEQ ID NO: 28), respectively.

In some embodiments, the anti-ToH1 antibody comprises a heavy chain variable domain (VH) comprising a sequence having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identity) to SEQ ID NO: 1, SEQ ID NO: 3, or SEQ ID NO: 5, and a light chain variable domain (VL) comprising a sequence having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identity) to SEQ ID NO: 2, SEQ ID NO: 4, or SEQ ID NO: 6. In some embodiments, the antibody comprises a heavy chain variable domain comprising a sequence having at least 90% sequence identity to SEQ ID NO: 1, SEQ ID NO: 3, or SEQ ID NO: 5, and a light chain variable domain comprising a sequence having at least 90% sequence identity to SEQ ID NO: 2, SEQ ID NO: 4, or SEQ ID NO: 6. In some embodiments, the antibody comprises a heavy chain variable domain comprising a sequence having at least 95% sequence identity to SEQ ID NO: 1, SEQ ID NO: 3, or SEQ ID NO: 5, and a light chain variable domain comprising a sequence having at least 95% sequence identity to SEQ ID NO: 2, SEQ ID NO: 4, or SEQ ID NO: 6.

In some embodiments, the anti-ToH1 antibody comprises a heavy chain variable domain having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identity) to SEQ ID NO: 1 and a light chain variable domain having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identity) to SEQ ID NO: 2. In some embodiments, the anti-ToH1 antibody comprises a heavy chain variable domain having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identity) to SEQ ID NO: 3 and a light chain variable domain having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identity) to SEQ ID NO: 4. In some embodiments, the anti-ToH1 antibody comprises a heavy chain variable domain having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identity) to SEQ ID NO: 5 and a light chain variable domain having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identity) to SEQ ID NO: 6.

In some embodiments, the antibody comprises a heavy chain variable domain comprising SEQ ID NO: 1 and a light chain variable domain comprising SEQ ID NO: 2. In some embodiments, the antibody comprises a heavy chain variable domain comprising SEQ ID NO: 3 and a light chain variable domain comprising SEQ ID NO: 4. In some embodiments, the antibody comprises a heavy chain variable domain comprising SEQ ID NO: 5 and a light chain variable domain comprising SEQ ID NO: 6.

In some embodiments, the VL and the VH are connected by a linker. In some embodiments, the linker comprises a series of repeating glycine residues. In some embodiments, the linker comprises GGGGSGGGGSGGGGSGGGGAS (SEQ ID NO: 10).

In some embodiments, the anti-ToH1 antibody is a nanobody. A nanobody refers to an antibody fragment derived from heavy-chain only IgG antibodies found in the Camelidae family. Heavy chain IgG (hcIgG) antibodies do not contain the CH1 region, but they retain the variable heavy domain, referred to in a heavy chain only antibody as “VHH”. Accordingly, a Fab fragment from a camelid antibody is also referred to herein as a “VHH”, a “single domain antibody”, or a “nanobody”, and refers to the fragment of a heavy-chain only antibody which consists of the variable domain (or a recombinant variable domain) of the heavy-chain only antibody.

In some embodiments, the anti-ToH1 antibody is a nanobody having at least 80% sequence identity with:

(SEQ ID NO: 29)
QVQLVESGGGLVQPGGSLRLSCAASGFTMGDYAIGWFRQVPGKER
EGLSYSTKNGIKAYADSVRDRFTISRENAKNTVYLQMNSLKPEDT
AVYYCAASTRFGGLLVSRYDYWGQGTQVTVSS

In some embodiments, the anti-ToH1 antibody is a nanobody having at least 80%, at least 81%, at least 82%, at least 83%, at least 84%, at least 85%, at least 86%, at least 87%, at least 87%, at least 89%, at least 90%, least 91%, at least 92%, at least 93%, at least 94%, at least 95%, at least 96%, at least 97%, at least 98%, or at least 99% identity to SEQ ID NO: 29.

In some embodiments, the anti-ToH1 antibody is a nanobody having at least 80% sequence identity with:

(SEQ ID NO: 30)
QLQLVESGGGLVQAGGSLRLSCAASGLTVSSYSMGWFRQAPGKER
EFVAAISRSGATINYGSSVQGRFMIARDDAKNTVNLQMNSLKPED
TAVYYCAARDRYSLVARAYEYWGQGTQVTVSS.

In some embodiments, the anti-ToH1 antibody is a nanobody having at least 80%, at least 81%, at least 82%, at least 83%, at least 84%, at least 85%, at least 86%, at least 87%, at least 87%, at least 89%, at least 90%, least 91%, at least 92%, at least 93%, at least 94%, at least 95%, at least 96%, at least 97%, at least 98%, or at least 99% identity to SEQ ID NO: 30.

### II. Methods of Antibody Generation

In some aspects, provided herein are methods of generating anti-ToH1 antibodies, including anti-ToH1 antibodies described above. In some embodiments, provided herein is a method of generating an anti-ToH1 antibody comprising sequentially immunizing a host with two or more unique peptides, and isolating antibodies generated in response to immunization. In some embodiments, each of the two or more unique peptides has the motif [S/T]xExPx[I/V], wherein each x is a variable amino acid. Each x may be any suitable amino acid. In some embodiments, the series of fixed amino acids in each peptide is unique. In some embodiments, the variable amino acids for each unique peptide are the same. In some embodiments, the variable amino acids for each unique peptide are not the same. In some embodiments, each peptide differs from one another peptide only at a single fixed amino acid position. In some embodiments, the two or more unique peptides comprises three unique peptides. In some embodiments, the two or more unique peptides comprises four unique peptides. In some embodiments, the two or unique peptides are independently selected from SxExPxI, SxExPxV, TxExPxI, and TxExPxV. In some embodiments, the host is vaccinated with three or more of SxExPxI, SxExPxV, TxExPxI, and TxExPxV. In some embodiments, the host is vaccinated with each of SxExPxI, SxExPxV, TxExPxI, and TxExPxV.

In some embodiments, the two or more unique peptides are independently selected from SAENPKI (SEQ ID NO: 42), SPEKPTV (SEQ ID NO: 36), TAEDPRI (SEQ ID NO: 41), and TSETPRV (SEQ ID NO: 33). In some embodiments, the host is immunized with three or more of SAENPKI (SEQ ID NO: 42), SPEKPTV (SEQ ID NO: 36), TAEDPRI (SEQ ID NO: 41), and TSETPRV (SEQ ID NO: 33). In some embodiments, the host is immunized with each of SAENPKI (SEQ ID NO: 42), SPEKPTV (SEQ ID NO: 36), TAEDPRI (SEQ ID NO: 41), and TSETPRV (SEQ ID NO: 33). The host may be immunized with the two or more unique peptides in any suitable order, with any suitable spacing between each immunization. In some embodiments, the host is immunized with one unique peptide on a given day. In some embodiments, the host is immunized with more than one unique peptide on a given day.

In some embodiments, the host is a transgenic animal. In some embodiments, the host is a cell. In some embodiments, the host is a mammalian cell.

### III. Diagnostic and Therapeutic Method

In some embodiments, anti-ToH1 antibodies, including anti-ToH1 antibodies described herein, are used in the diagnosis, treatment, and/or prevention of microbial disease and/or chronic inflammatory disease. In some embodiments, the chronic inflammatory disease is a microbe-associated chronic inflammatory disease. In some embodiments, anti-ToH1 antibodies are used for the diagnosis and/or treatment of disease caused or exacerbated by virulent microorganisms. In some embodiments, anti-ToH1 antibodies are used to treat or prevent microbial virulence.

In some aspects, provided herein are methods of diagnosing microbial disease and/or chronic inflammatory disease in a subject. In some embodiments, methods of diagnosing microbial disease and/or chronic inflammatory disease in a subject comprise determining levels of target of HGMB1 (ToH1) in a sample obtained from a subject, and determining that the subject has a microbial disease when the level of ToH1 in the sample are equal to or above a threshold value. In some embodiments, levels of ToH1 in the sample are determined by performing an immunoassay (e.g. ELISA). In some embodiments, determining levels of ToH1 in the sample comprises contacting a sample obtained from a subject with an antibody that binds to target of HGMB1 (ToH1), including an anti-ToH1 antibody described herein, and detecting the antibody in the sample. In some embodiments, the anti-ToH1 antibody is labeled, such as with a fluorescent label or tag, and the label can be detected by a suitable assay in order to determine the level or amount of ToH1 in the sample. In some embodiments, the threshold value is determined based upon levels of ToH1 observed in samples obtained from subjects not having a microbial infection. In some embodiments, the method further comprises administering a suitable treatment to a subject when levels of ToH1 in the sample are indicative of microbial infection in the subject. In some embodiments, treatment comprises an anti-ToH1 antibody. In some embodiments, treatment comprises an anti-microbial agent, such as an antibiotic, antiviral, antifungal, anti-inflammatory agent, or a combination thereof.

In some embodiments, provided herein are methods of treating or preventing microbial disease and/or chronic inflammatory disease in a subject, the method comprising providing to the subject an antibody that binds to target of HGMB1 (ToH1). In some embodiments, the subject is determined as having a microbial disease by performing a diagnostic method provided herein involving contacting a sample obtained from the subject with an anti-ToH1 antibody and measuring the antibody in the sample.

In some embodiments, anti-ToH1 antibodies are conjugated to a therapeutic agent (e.g. antimicrobial agent, anti-inflammatory agent, or other agent). In some embodiments, anti-ToH1 antibodies are administered in combination with a therapeutic agent (e.g., antimicrobial agent, anti-inflammatory agent, or other agent). In some embodiments, the anti-ToH1 antibody is formulated as a composition (e.g. a pharmaceutical composition) comprising the anti-ToH1 antibody and a suitable carrier (e.g. pharmaceutically acceptable carrier). The antibody or composition comprising the antibody can be administered to the subject by any suitable route. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. In some embodiments, the antibody or composition comprising the antibody is administered parenterally (e.g. subcutaneously, intravenously).

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the active agents of the formulation.

Dosing is dependent on severity and responsiveness of the disease state or condition to be treated, with the course of treatment lasting from several days to several months, or until a cure is effected or a diminution of the disease state is achieved. In some embodiments, treatment is administered in one or more courses, where each course comprises one or more doses per day for several days (e.g., 1, 2, 3, 4, 5, 6) or weeks (e.g., 1, 2, or 3 weeks, etc.). In some embodiments, courses of treatment are administered sequentially (e.g., without a break between courses), while in other embodiments, a break of 1 or more days, weeks, or months is provided between courses. In some embodiments, treatment is provided on an ongoing or maintenance basis (e.g., multiple courses provided with or without breaks for an indefinite time period). Optimal dosing schedules can be calculated from measurements of drug accumulation in the body of the patient. The administering physician can readily determine optimum dosages, dosing methodologies and repetition rates.

In some embodiments, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues.

In some embodiments, the microbial disease (e.g. the disease diagnosed, treated, or prevented in the subject) comprises a microbial infection. The microbial infection may be caused by any microbe, including bacterial infections, viral infections, fungal infections, and/or protozoal infections. In some embodiments, the microbial infection is a bacterial infection. Nonlimiting examples of exemplary infections include gastrointestinal infections (cholera, salmonellosis, Clostridium difficile infections, listeriosis), sexually transmitted diseases (chlamydia, syphilis), meningococcal disease, dermatological infections (staphylococcal infections), and lung infections (pertussis, pneumonia).

In some embodiments, the chronic inflammatory disease is a microbe-associated chronic inflammatory disease. In some embodiments, the chronic inflammatory disease is inflammatory bowel disease, rheumatoid arthritis, non-alcoholic fatty liver disease, type II diabetes, urinary tract infections, pneumonia, or sepsis. In some embodiments, the chronic inflammatory disease is inflammatory bowel disease. The term “inflammatory bowel disease” refers to disorders involving chronic inflammation of the tissues of the digestive tract and includes Crohn's disease and ulcerative colitis (UC).

In some embodiments, the subject is a vertebrate. In some embodiments, the subject is a bird (e.g. poultry, such as chickens), a reptile, or a mammal. In some embodiments, the subject is a human. In some embodiments, the subject has a deficiency in HMGB1.

### EXPERIMENTAL

The following examples are provided in order to demonstrate and further illustrate certain preferred embodiments and aspects of the present invention and are not to be construed as limiting the scope thereof.

### Example 1

### Methods

### Mice

All mice used in this study were on a C57BL/6 genetic background. Specific pathogen-free (SPF) mice were purchased from Jackson Labs. Germ-free (GF) mice were maintained under gnotobiotic conditions prior to euthanasia. Hmgb1fl/fl (WT) and Hmgb1fl/fl, Vil-CRE (ΔIEC) mice were created. Mice were maintained under SPF conditions, apart from the GF mice. All animal experiments were performed at least two and in most instances three independent times using both age and sex-matched mice. When possible, littermate controls were used. Mice were between 6 and 12 weeks of age and both sexes were used for all experiments. Mice were housed under a 12-hour light/dark cycle and fed standard laboratory chow.

### Bacteria Culture

All E. coli strains were cultured under fimbriae inducing conditions. Strains were passaged twice under static conditions in nutrient broth at 37° C. or passaged once on a nutrient agar plate incubated overnight at 37° C. unless otherwise indicated. Media consisted of 0.5% Bacto Peptone, 0.3% Beef Extract, 0.5% NaCl and 1.5% agar (if needed), pH 6.8. BW25113 mutant construction Full-length FimH was PCR amplified from BW25113 and cloned into the pGex6-1 vector using restriction digest cloning and the BamHI and XhoI restriction sites. Mutation of the ToH1 sequence in FimH was done using two rounds of PCR. Mutation at desired region was introduced in the first round of PCR. Two fragments were generated using primer pairs 1) FimH-For and FimH-Mut1 and 2) FimH-Rev and FimH-Mut2. These two fragments were gel purified, combined, and were used as template for second round of PCR. FimH-For and FimH-Rev were used in the second round of PCR. The products were gel purified, digested by BamHI and XhoI, and cloned into BamHI and XhoI digested pGex6-1 vector. Wild type and mutant plasmids were verified by ability of transformed cells to grow on selection antibiotics and Sanger sequencing. E. coli knocked out for FimH (ΔFimH) were transformed with either plasmids containing wild type FimH (ΔFimHWT) or FimH mutated in ToH1 ((ΔFimHMut). See Table 1 for oligonucleotide information.

TABLE 1

| Oligonucleotides | Source | Name |
|---|---|---|
| 5ATTO647NN/GCT GCC TCC CGT AGG AGT (SEQ ID NO: 43) | IDT | ATTO647N |
| | | NEUB338 |
| | | probe |
| GGCATTCGGGCTCCTTTCTT (SEQ ID NO: 44) | Invitrogen | Mouse |
| | | Muc1 F |
| TGGAGTGGTAGTCGATGCTAAG (SEQ ID NO: 45) | Invitrogen | Mouse |
| | | Muc1 R |
| AGGGCTCGGAACTCCAGAAA (SEQ ID NO: 46) | Invitrogen | Mouse |
| | | Muc2 F |
| CCAGGGAATCGGTAGACATCG (SEQ ID NO: 47) | Invitrogen | Mouse |
| | | Muc2 R |
| GCCGTGAATTGTATGAACGGA (SEQ ID NO: 48) | Invitrogen | Mouse |
| | | Muc3 F |
| CGCAGTTGACCACGTTGACTA (SEQ ID NO: 49) | Invitrogen | Mouse |
| | | Muc3 R |
| AGGTCGGTGTGAACGGATTTG (SEQ ID NO: 50) | Invitrogen | Mouse |
| | | GAPDH F |
| TGTAGACCATGTAGTTGAGGTCA (SEQ ID NO: 51) | Invitrogen | Mouse |
| | | GAPDH F |
| ACTCCTACGGGAGGCAGCAG (SEQ ID NO: 52) | IDT | 16S F |
| ATTACCGCGGCTGCTGG (SEQ ID NO: 53) | IDT | 16S R |
| GCTGGTGGTAGGAAATGGATA (SEQ ID NO: 54) | IDT | FimH F |
| CTTATGGCGGCGTGTTATCT (SEQ ID NO: 55) | IDT | FimH R |
| GAGCAAGCGGACCTCATAAA (SEQ ID NO: 56) | IDT | 16S F |
| GGCATTCTGATCCACGATTACTA (SEQ ID NO: 57) | IDT | 16S R |
| GGCCGGGATCCAAACGAGTTATTACCCTGTTTGCTGTAC (SEQ ID | IDT | FimH-F |
| NO: 58) | | |
| GCGCGCTCGAGTTATTGATAAACAAAAGTCACGCCAATAATC (SEQ | IDT | FimH-R2 |
| ID NO: 59) | | |
| AGCGCGGGCCGTAGCGCTTGCGGTAGGAAATGGATAGCTACTGCC | IDT | FimH-Mut1 |
| (SEQ ID NO: 60) | | |
| GCAAGCGCTACGGCCCGCGCTGTTTATAATTCGAGAACGGATAAGC | IDT | FimH-Mut2 |
| CG (SEQ ID NO: 61) | | |

### SWW33 Mutant Construction

Approximately 1 kb homologous recombination arms flanking the sequence to be mutated/deleted (FimH), were PCR amplified from the genomic DNA of WT SWW33. These two arms were then fused together directly or fused to the mutated sequence through PCR. Using TAKARA infusion kit, the PCR-generated fragment was ligated to a suicide plasmid containing kanamycin resistance cassette, R6K origin, RP4 mob, and sacB gene. This suicide plasmid was transformed into E. coli S17-1λpir through electroporation, and then transformed into E. coli SWW33 through conjugation. The successful transconjugants were obtained through plating the conjugation mixture on LB agar plates supplemented with kanamycin and ampicillin. The SWW33 strain that was transformed has an ampicillin resistant plasmid prior to conjugation. The purified transconjugants were then plated on LSW61 agar plates supplemented with 10% sucrose to select for the successful knock-in mutants, which were then purified and verified through PCR, Sanger sequencing, and their ability to grow in the presence of ampicillin but not kanamycin. The composition of the LSW agar plates (per liter) was 10 g tryptone, 5 g yeast extract, 5 mL glycerol, 0.4 g NaCl, and 20 g agar. See extended Table 1 for oligonucleotide information.

### Immunostaining

Murine colon tissue was obtained from 8 to 12-week-old mice. Human colon samples were residual tissue from colonic resections performed for clinical care of disease (obtained under an existing IRB). Samples with no history of inflammatory bowel disease were used as controls. All samples were fixed in methyl-Carnoy's fixative (60% methanol, 30% chloroform, 10% glacial acetic acid) for 3 hours at 4° C. prior to transfer to 70% ethanol. Paraffin-embedded sections were deparaffinized to water and slides underwent antigen retrieval in 10 mM sodium citrate, 0.05% Tween 20 pH 6.0 using a steamer for 20 minutes and were allowed to cool for 1 hour. They were blocked in serum-free protein block (Agilent, Dako, X0909) and stained with either 10 mg/mL Dylight 594 conjugated Ulex Europaeus Agglutinin I (UEA I), 0.17 mg/mL anti-HMGB1 (Abcam), 0.874 mg/mL anti-FimH polyclonal antibody (custom antibody produced by Genscript) or 0.96 mg/mL anti-Muc2 (Novus). 1 mg/mL Alexa Fluor 647 donkey anti-rabbit IgG was used as a secondary if needed. Tyramide SuperBoost (Thermo Fisher Scientific) was used to amplify signals from anti-FimH polyclonal antibody (custom antibody produced by Genscript). Slides were counterstained using 10 mg/mL bisBenzimide H 33258 dissolved in TBS for 20 minutes in the dark at room temperature then cover slipped. For peptide inhibition staining, 0.85 mg/mL of human HMGB1 peptide (Abcam), or 9 ug/mL of full-length FimH protein (antigen for custom antibody production, Genscript) was used. All immunofluorescent sections were analyzed using either a widefield fluorescent microscope (Keyence BZ-800) or an inverted confocal microscope (40×/1.25 oil objective on a Leica TCS-SP8-AOBS inverted confocal microscope (Leica Microsystems, GmbH, Wetzlar, Germany)).

### Enzyme-Linked Immunoassay for HMGB1

Samples used for measuring HMGB1 protein levels in the stool and colonic mucus were homogenized in cell lysis buffer (Cell Signaling) containing complete protease inhibitor (Roche) and 100 mM PMSF. After centrifugation at top speed for 15 minutes, supernatants were collected and assayed for protein concentrations via BCA (Thermo Scientific). Samples were analyzed using an HMGB1 detection kit per the manufacturer's instructions (Chondrex).

### Immunoblot Analysis

Bacterial and mucus samples were boiled in Laemmli buffer containing 10% b-mercaptoethanol. All samples were separated using NuPage Bis-Tris gels with MOPS or MES running buffers. Proteins were transferred to PVDF membranes and probed with FimH (Sokurenko Lab), GAPDH (Cell Signaling), 0.17 mg/mL antiHMGB1 (Abcam), 0.6 mg/mL mouse anti-FimH (Sokurenko (mouse samples) or custom antibody produced by Genscript (bacterial samples), 0.1 mg/mL anti-GroEL (Enzo), or 680-labeled streptavidin overnight in 5% w/v non-fat dry milk Omniblock in 0.1% PBST or LI-COR Intercept PBS blocking buffer at 4° C.62. After incubation and washing, 0.08 mg/mL of HRP conjugated anti-mouse or anti-rabbit, 0.2 ug/mL 680RD Streptavidin (LICOR), or 0.2 ug/mL 800RD (LI-COR) anti-mouse secondary antibody was added to the blot and incubated for one hour. After washing, chemiluminescence signal was captured either by film or by using a Bio-Rad ChemiDoc MP and fluorescent signal was detected using an LI-COR Odyssey CLX machine and LI-COR Acquisition software.

### Fluorescent In-Situ Hybridization (FISH) to Detect Bacteria

Paraffin-embedded sections were deparaffinized to water. Permeabilization solution (5 mg/mL lysozyme, 0.05 M EDTA, 0.1 M Tris pH 7.4, PBS) was applied to tissue and incubated at 37° C. for 20 minutes followed by a PBS wash. Slides were incubated in a hybridization solution (0.9 M NaCl, 0.02 M Tris pH 7.4, 0.005 M EDTA, 1% v/v Triton X-100, 35% deionized formamide, 0.1% w/v BSA, water; pre-warmed to 46° C.) for 1 hour in a hybridization oven at 46° C. A universal bacterial probe (EUB338 modified with a 5′ ATTO 647N dye) was denatured in the hybridization solution heated to 95° C. for 2 minutes. The slides returned to the hybridization oven covered with 0.1 M probe in hybridization solution for 2.5 hours at 46° C. Slides were washed three times in stringency wash buffer 1 (2×SSC (300 mM NaCl, 30 mM Sodium Citrate, pH 7.0 pre-warmed to 46° C.)). A secondary wash was performed three times in stringency wash buffer 2 (0.1×SSC (15 mM NaCl, 1.5 mM Sodium Citrate, pH 7.0)) at room temperature and rinsed in PBS. Tissues were counterstained with 10 mg/mL bisBenzimide H 33258 dissolved in TBS for 20 minutes in the dark at room temperature and coverslipped. The distance between the bacteria and the epithelia was performed using ImageJ analysis software.

### Bacterial Flow Cytometry

HMGB1 Binding to E. coli

Bacterial preparations of 3.2×10 8 (O.D. 0.4) E. coli were made from an overnight agar culture and incubated with 3 mM HMGB1 recombinant protein containing a HIS tag (Abclonal) or an hFc tag (Sino Biological) and reconstituted in 1 mM DTT, 1 mM EDTA, PBS buffer for two hours at 37° C. Cells were fixed with 4% PFA and blocked with 1% BSA in PBST for 1 hour. An unconjugated Anti-HIS antibody (Genscript) was added (5.0 mg/mL) and incubated overnight at 4° C. The following day, cells were washed in PBS and secondary antibody (0.25 mg/test PE-Cy7) added for 1 hour at room temperature. Samples incubated with hFc tagged HMGB1 were incubated with PE anti-human IgG Fc recombinant antibody (0.25 mg/test) for 1 hour at room temperature. Finally, cells were stained with 1 mM SYTO9. Bacterial suspensions were analyzed using an Attune N×T flow cytometer (Invitrogen) with FlowJo software (Tree Star).

### HMGB1 Binding to ToH1

Bacterial preparations of 3.2×108 (O.D. 0.4) E. coli were made from an overnight agar culture and incubated with 3 mM HMGB1 (R&D systems) diluted with protein block buffer (Dako) for 2 hours. Bacteria were centrifuged and washed three times with PBS, fixed with 4% PFA for 10 minutes and washed with PBS for three more times. Samples were incubated with anti-FimH (Sokurenko) or rabbit anti-HMGB1 (Abcam) incubated overnight at 4° C.62. Samples were washed and Invitrogen Alexa a10040, 1:1500 was added. Samples were analyzed using an Image Stream MarkII from Amnis (Luminex).

### Intestinal Epithelial Cell Adhesion Assay

Caco2BBe1 cells were seeded onto 0.4 um inserts of a 6.5 mm Transwell (Corning) at 250,000 cells per well and allowed to form a polarized monolayer. 72 hours prior to the adhesion assay, 100 ug/mL kifunensine (Cayman Chemicals) was supplemented to 1×DMEM+10% FBS. Bacterial preparations grown from an overnight nutrient agar culture (1.6×108 (O.D. 0.2) E. coli per well) were suspended in serum-free 1×DMEM and incubated with 3 mM HMGB1 (R&D), 100 mM mannose (Sigma-Aldrich), or buffer vehicle (1 mM DTT, 1 mM EDTA, PBS) for 1 hour at room temperature. The apical media from confluent Caco2BBe1 cells were removed and cells washed three times with serum-free 1×DMEM. Treatments were added to wells and cells incubated for 1 hour at 37° C. Cells were washed three times by adding serum-free 1×DMEM and letting the plate shake for 1 minute at 100 RPM between washes. Cells were dislodged with the addition of 0.1% Triton X-100 in PBS, incubated for 5 minutes at room temperature then scraped and collected. Serial dilutions were performed and plated on LB agar plates incubated overnight at 37° C. and counted the following day. Serial dilutions of the input E. coli were also plated and used to normalize the recovered E. coli.

qRT-PCR

RNA isolation was performed on colonic mucosal scrapings or bacterial lysates using TRIzol (Life Technologies) following the standard extraction procedure. Gene expression was determined using SYBR Green Master Mix (Bio-rad) and a Roche LightCycler 480 System. See Table 1 for oligonucleotide information.

qPCR

DNA isolation was performed on mucosal scrapings or bacterial lysates using DNeasy Powerlyzer Microbial kit per the manufacturer's instructions. Quantitative PCR was performed using PowerUp SYBR Green Master Mix (Applied Biosystems) in a CFX96 Touch Real-Time PCR system. See Table 1 for oligonucleotide information.

### Red Blood Cell Agglutination

Red blood cell agglutination assays were performed as previously described. Briefly, serial dilutions of E. coli were incubated with a 3% solution of washed erythrocytes in a round bottom 96 well plate. The wells were mixed by shaking the plate. The plate was incubated at room temperature and imaged 1 hour following shaking. The lowest bacterial number to achieve agglutination was used for the HMGB1 inhibition assay. For the inhibition assay, E. coli were incubated with serial dilutions of HMGB1 recombinant protein (R&D systems) reconstituted in 1 mM DTT, 1 mM EDTA, PBS buffer and a 3% solution of washed erythrocytes. The wells were mixed by shaking the plate. The plate was incubated at room temperature and imaged 1 hour following shaking.

### Bacterial Aggregation Assays

E. coli Aggregation

Bacteria expressing green fluorescent protein (GFP) were grown statically under antibiotic selection for two overnight passages before experimental treatment. The final overnight culture was adjusted to an O.D. of 0.4 and 100 mL was centrifuged into a pellet at 5000 RPM for 5 minutes. The supernatants were discarded, and cells were resuspended in TBS with 10 mM CaCl2. Bacteria were treated with either 3 mM recombinant HMGB1 (R&D systems) or buffer in a 50 mL reaction at 37° C. for 2 hours. Cells were carefully dispensed onto glass coverslips, covered with a 0.15% agarose pad, and imaged using fluorescent microscope. The remaining bacterial samples were analyzed using an LSRFortessa (BD) with FlowJo software (Tree Star) following fixation with 4% PFA.

### Colonic Community Microbiota Aggregation

Colons from SPF C57BL/6J mice were excised and fileted open. An inoculating loop was used to scrape the contents into a 1.5 mL centrifuge tube containing 1 mL PBS. After centrifugation at 400 G for 5 minutes, the supernatant was strained using a 70 mm cell strainer. The flow through was washed twice with PBS and once with TBS containing 10 mM CaCl2 at 10,000×G for 2 minutes. After the third wash the sample was resuspended in 500 mL TBS/CaCl2. The sample was split into 100 uL aliquots and centrifuged at 10,000×G for 2 minutes. rHMGB1 (R&D systems) was fluorescently labeled with AlexaFluor 647 labeling kit according to the manufacturer's protocol. Bacteria were resuspended in either 3 mM of labeled rHMGB1 or buffer containing 1 mM SYTO 9 and incubated at 37° C. for 2 hours. Cells were carefully dispensed onto glass coverslips, covered with a 0.15% agarose pad, and imaged under fluorescence.

### Recombinant FimH Lectin Domain Production

Recombinant FimH lectin domain protein was produced in HM125 E. coli using plasmids. Purification of the K12 FimH lectin domain was performed as previously described. FimH binding to mannose FimHLD (40 ug/mL) was incubated with serial dilutions of HMGB1 recombinant protein (R&D systems) for 1 hour at room temperature. A mannose coated 96-well plate was washed with 200 uL of 10 mM Tris+0.05% tween pH 8.0, then the samples were applied to the plate. The plate was sealed and incubated overnight at 4° C. The plate was washed with PBST (0.1%) 4× and blocked with 1% BSA in PBST (0.1%) for 1 hour at room temperature. Then the plate was washed with PBST (0.1%) 4× and the primary anti-HIS antibody was diluted in 0.1% BSA in PBST at a concentration of 5 ug/mL and added to the plate. This was incubated for 1 hour at room temperature. The plate was then washed with PBST (0.1%) 4× and the secondary anti-mouse HRP antibody was added to the plate at 0.16 ug/mL diluted in 0.1% BSA in PBST. The plate was incubated for 30 minutes at room temperature. The plate was washed with PBST (0.1%) 4× and TMB solution was added to the wells. The reaction was quenched with 0.5M sulfuric acid and read at 450 nm on a spectrophotometer.

E. coli Killing by HMGB1

Bacterial preparations of 6.4×108 (O.D. 0.8) E. coli were made from twice passaged nutrient broth cultures and incubated with 3 uM HMGB1 recombinant protein (R&D systems) reconstituted in 1 mM DTT, 1 mM EDTA, PBS buffer for 2 hours at 37° C. Following incubation, cells were serially diluted, plated onto LB agar plates and colonies were counted the following day after incubation at 37° C.

FimH Expression in E. coli Exposed to HMGB1

Bacterial preparations of 3.2×108 (O.D. 0.4) E. coli were made from an overnight agar culture and incubated with 3 μM HMGB1 recombinant protein (R&D systems) reconstituted in 1 mM DTT, 1 mM EDTA, PBS buffer for 2 hours at 37° C. RNA was isolated using TRIzol (Life Technologies) according to manufacturer's instructions. Fim switch PCR assay After shaking at 255 rpm for two overnight passages in broth, E. coli preparations of 6.4×108 cells/mL (O.D. 0.8) were made. The bacteria were treated with 1/100 dilution of conditioned organoid media with (WT) or without (ΔIEC) HMGB1 for 18 hours at 37° C. statically. Pilot assays with media derived from small and large intestine derived organoids showed similar results, so additional assays were performed with small intestinal derived organoids since the media contained fewer additives. The conditioned organoid media collected from small intestinal organoids before passaging comprised of Advanced DMEM/F12 supplemented with 1× LGlutamine (Life Technologies), 10 mM HEPES buffer (Life Technologies), 1× penicillin and streptomycin (Life Technologies), 1× N2 supplement (Life Technologies), 1× B-27 Supplement Minus Vitamin A (Life Technologies), 50 ng/ml murine Epidermal Growth Factor (Peprotech), 100 ng/mL Noggin (Peprotech), 1 μM Jagged 1 (Anaspec), 10 nM Y-27632 (Cayman Chemical Company), and 100 ng/mL R-spondin 1 (Peprotech). This was concentrated using a 3 kDa MWCO filter insert with PBS as the diluent. Bacterial DNA was extracted using DNeasy PowerLyzer Microbial Kit following the manufacturer's protocol. PCR amplification was performed as previously described. Phase-ON and Phase-OFF fmS PCR product band intensities were standardized against the fsZ amplification product using ImageJ software. See Table 1 for oligonucleotide information.

### Quantification of FimH Positive Bacteria in IF Images

Image-Pro Plus 7.0 was used for image operations and measurements. Images were spatially calibrated to units of micrometers and processed as gray values. Background subtraction commensurate with secondary antibody negative controls were applied across the dataset. Semi-automatic counting was performed by manually setting the intensity range overlapped with images. Objects and measurements were collected across five image fields per sample. Measurements of each object were taken to sort and describe post hoc. Two independent people manually applied the intensity ranges with little deviation and similar results. The five image counts were averaged to represent each sample in units of objects per field. Quantification of surface associated HMGB1 in IF images Leica Application Suite (version 3.7.5 or higher) was used for measuring fluorescence intensity in images gathered on a Leica TCS-SP8-AOBS inverted confocal microscope. Regions of interest were drawn around representative surface epithelium and maximized area. Mean fluorescent values (RFU/um\hat{ }2) were gathered in each image. In background subtraction from the secondary antibody, only negative controls were applied across the dataset. An average of five image values were used to represent each sample. Two independent people identified representative regions of interest (surface epithelium) to collect fluorescent values with similar results.

### Murine Mucus Isolation

Mucus isolation was performed as previously described. Murine mucosal scrape Colons from mice were excised, fileted open and the contents were washed off. One mL of PBS was added to the colonic tissue in a 1.5. mL centrifuge tube and vortexed vigorously for 2 minutes. The tissue was removed and the mucus containing mixture was centrifuged at top speed for 5 minutes. The semi-transparent mucus layer on top of the dark pellet was removed and resuspended in 1 mL of PBS. 20 mL was saved for BCA. The remainder was centrifuged again and the mucus containing pellet was solubilized in Laemmli buffer for immunoblotting at sample buffer for ELISA.

### Mucus Invasion Assay

Bacteria expressing GFP were grown statically overnight in broth for two passages and made into preparations of 8×108 cells/mL (O.D. 1.0). These E. coli preparations were used to fill the middle channel of a chemotaxis μ-Slide (Ibidi). Mucus isolated from WT and DIEC mice were added to opposing reservoirs of the same chamber and incubated at 37° C. for 1 hour. Five representative images of fluorescent bacteria invading mucus samples were captured along the leading mucosal edge, proximal to the middle channel of the chamber. These assays were performed at least three times with different bacterial preparations. Any and all image processing settings to reduce background signal were applied across the whole dataset. Measuring the percent relative fluorescence in mucus was performed by using the ImageJ analysis software. After spatially calibrating the image set units to known micrometers, a region of interest was applied beginning at the leading mucosal edge, spanning its full length, and approximately 300 mm deep in each image; approximately totaling 170,000 μm²; or the maximum area common to all images. Integrated density values were collected and divided by the area of the region of interest (ROI) to account for possible variances upon re-drawing ROIs. Those mean fluorescence values were averaged between the five images and used to represent each sample within the chamber slide. Given that bacteria had an equal probability of traveling in either well's direction, the mean fluorescence of each well was compared to the other as a proportion of the total fluorescence of the reaction chamber. Two independent investigators identified regions of interest to collect fluorescent values with similar results.

### Sulfo-SBED Label Transfer

The label transfer reaction was performed at previously described68. Briefly, rHMGB1 (R&D Biosystems) was treated as the “bait protein” and was reconstituted to 0.2 mg/mL in sterile PBS. Sulfo-SBED was calculated to be added at a 3-molar excess over the purified HMGB1. Sulfo-SBED was dissolved in DMSO to 10 mg/ml and added to the rHMGB1. The reaction was incubated at room temperature for one hour protected from light. Then labeled protein was added to a desalting column (Thermo Fisher #89849) equilibrated with 50 mM HEPES, 150 mM NaCl; pH 7.3 to remove the excess crosslinker and leave the labeled purified HMGB1 in the proper reaction buffer. Sulfo-SBED labeled HMGB1 and unlabeled FimHLD were allowed to incubate together for 2 hours at room temperature protected from light to ensure interaction. The molar ratio used of FimH:HMGB1 was 2.2 mM FimH: 4 mM HMGB1 and was performed in a 50 ml reaction. To crosslink the samples, reactions were moved to a clear 96-well polypropylene low binding plate placed 5 cm from the UV light source (Stratalinker) and exposed to 538 nm wavelengths for 8 minutes on ice. Crosslinked reactions were moved into fresh tubes with the addition of a DTT sample buffer to a final DTT concentration of 100 mM to complete disulfide bond reduction and transfer the biotin label. Samples were heated to 70° C. for 10 minutes and analyzed by immunoblot.

### Microbiome Studies

Microbiome sampling: Littermate mice of both sexes and genotypes were separated into group housing by genotype at weaning. Soiled bedding from mice of both genotypes was collected, mixed, and distributed across all of the cages at the time of separation. At 20 weeks of age, fresh stool samples were collected and the mice were sacrificed for collection of mucosal scrapings. Samples were submitted to the Environmental Sample Preparation and Sequencing Facility at Argonne National Laboratory for analysis of 16S rRNA. Bioinformatics: Individual fastq files without non-biological nucleotides were processed using Divisive Amplicon Denoising Algorithm (DADA) pipeline. The output of the dada2 pipeline (feature table of amplicon sequence variants (an ASV table)) was processed for alpha and beta diversity analysis using phyloseq, and microbiomeSeq (http://www.github.com/umerijaz/microbiomeSeq) packages in R. Alpha diversity estimates were measured within group categories using estimate richness function of the phyloseq package. Multidimensional scaling (MDS, i.e., PCoA) was performed using Bray-Curtis dissimilarity matrix between groups and visualized using the ggplot2 package. As appropriate, we adjusted for multiple comparisons using the BH FDR method while performing multiple testing on taxa abundance across groups. An analysis of variance across the groups for α-diversity was performed. Permutational multivariate analysis of variance (PERMANOVA) was performed on all principal coordinates obtained during PCoA. Linear regression (parametric) and Wilcoxon (non-parametric) were performed on ASV abundances vs metadata variable levels (e.g., diet components) using R base functions.

### Results

HMGB1 is Released into Colonic Mucus in Response to the Gut Microbiota

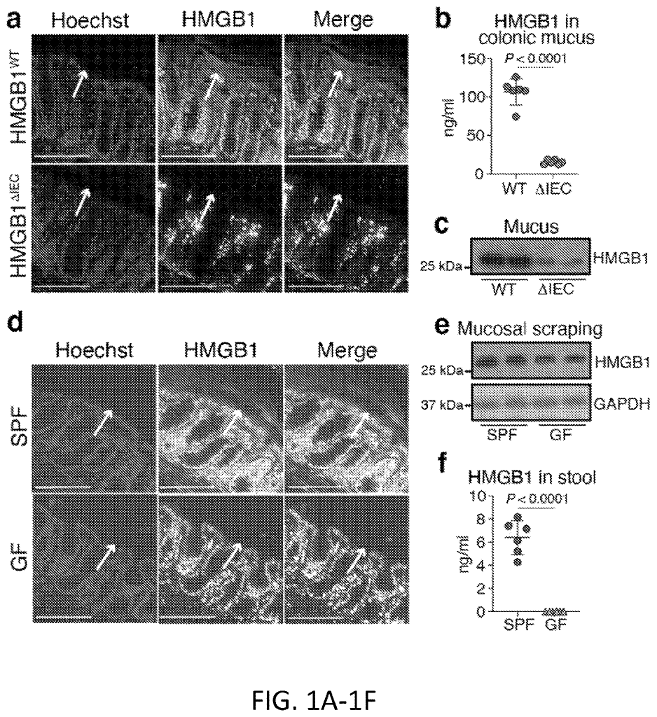

HMGB1 was highly concentrated at the luminal surface of the mouse colon in the tight, epithelial-associated mucus layer (FIG. 1a, FIG. 5). In littermate ΔIEC mice, HMGB1 was absent from IEC bodies and profoundly decreased in mucus, suggesting that IEC are the primary source of HMGB1 in colonic mucus (FIG. 1b, c). HMGB1 protein was likewise detectable in IEC from C57BL/6 germ-free (GF) mice and the levels appeared only mildly decreased compared to specific pathogen free (SPF) C57BL/6 mice (FIG. 1d, e). However, the presence of HMGB1 in the gut lumen was dependent on microbiota as HMGB1 staining was absent from the colon surface and HMGB1 protein was undetectable in stool from GF mice (FIG. 1d, f). HMGB1 was assayed in stool because GF mice produce very little colonic mucus. Taken together, these data show that HMGB1 is released from IEC into the colonic mucus in response to the gut microbiota.

HMGB1 Prevents Bacterial Invasion into the Inner Mucus Layer of the Colon

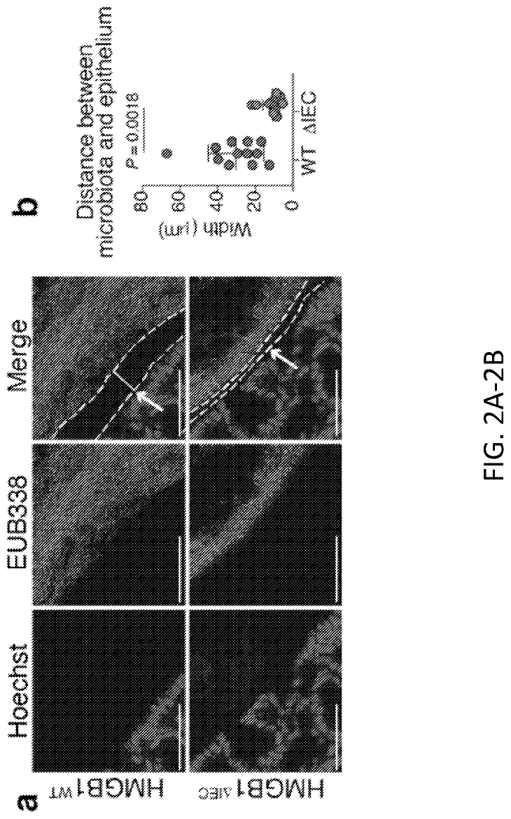

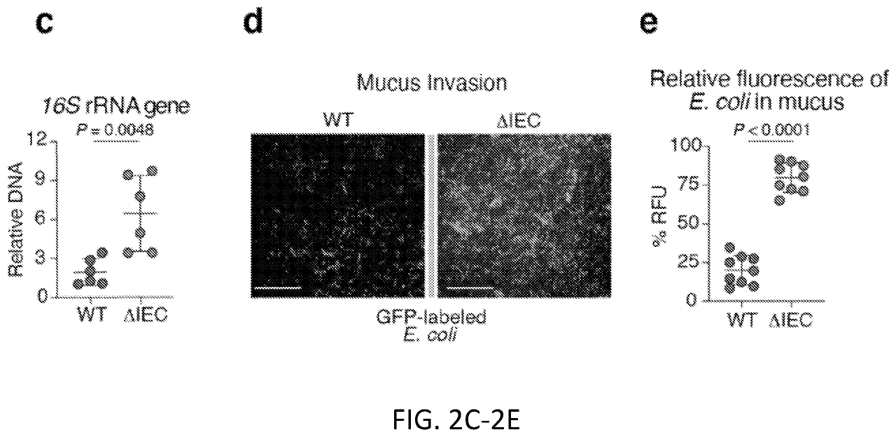

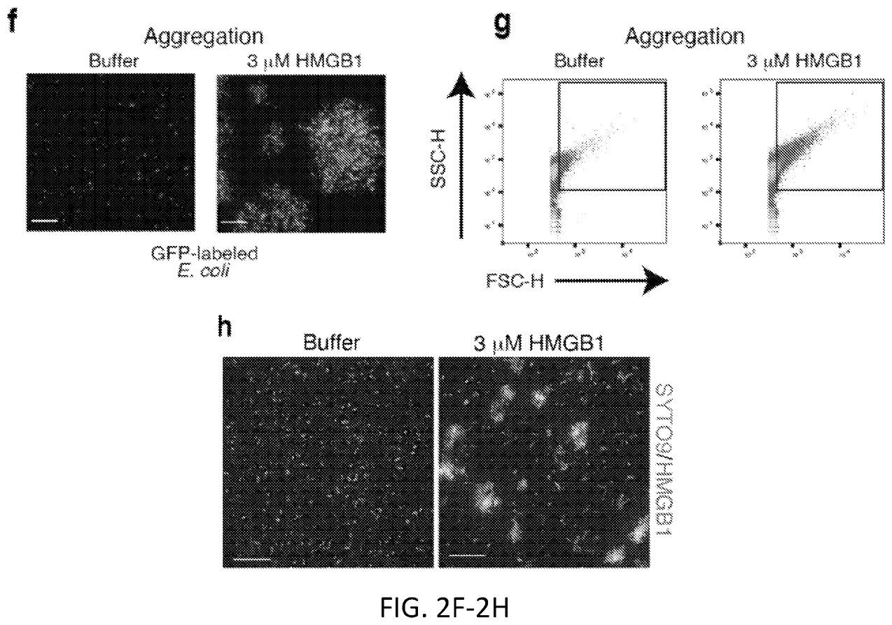

The presence of HMGB1 in the gut lumen suggested that it could affect the gut microbiota, as such the influence of HMGB1 on the composition and behavior of the gut bacterial community was next evaluated. The normal colon boasts a diverse, abundant microbial community that is physically separated from the epithelial surface by the inner mucus barrier. In mice lacking mucosal HMGB1, this physical separation was essentially lost leading to close proximity of the microbial community to host tissue along with an increase in bacterial DNA associated 3 with host tissue (FIG. 2a, b, c). The change in microbial biogeography was not due to a decrease in mucus production in ΔIEC mice. HMGB1 labeled microbes in the gut lumen, which led us to assess whether HMGB1 has direct effects on the microbiota (FIG. 5). The mucosal-associated microbial community did differ between WT and ΔIEC mice (FIG. 5b, c). Most notably, the taxonomic distinction normally present between stool and mucosal-associated bacteria was diminished in ΔIEC mice, reinforcing that HMGB1 is responsible for excluding microbiota from the inner mucus layer (FIG. 5D). However, taxonomic differences in mucosal-associated bacteria between the mouse genotypes were primarily appreciated at the strain level, suggesting that HMGB1 does not exert strong selection pressure on gut bacteria (FIG. 5E). Loss of HMGB1 might allow normally commensal microbes to penetrate colonic mucus. Mucus is thought to primarily act as a physical anti-microbial barrier in the colon. The most abundant protein in intestinal mucus, Mucin 2 (Muc2), is a large, heavily glycosylated protein that oligomerizes to form a dense meshwork that blocks bacterial movement. Additionally, oligosaccharides attached to Muc2 are the same oligosaccharides that bacterial adhesins bind to on the surface of host cells, so they serve as decoy adhesion sites and arrest movement through mucus. Microbial invasion into mucus was limited when HMGB1 was present in mucus (FIG. 2d, e). E. coli was chosen for these studies because it is a well-characterized gut commensal organism that has been associated with colitis in animal models and in human inflammatory bowel disease (IBD) patients. Exposure to HMGB1 also caused E. coli and complex microbiota to aggregate (FIG. 2f, g, h). These findings suggest that HMGB1 traps bacteria that enter colonic mucus and blocks their access to adhesion targets. Microbes coming into contact with HMGB1 are aggregated and prevented from migrating through the mucus and interacting with the epithelial surface of the colon.

### HMGB1 Binds and Inactivates the Bacterial Adhesin FimH Through an Evolutionarily Conserved Amino Acid Sequence

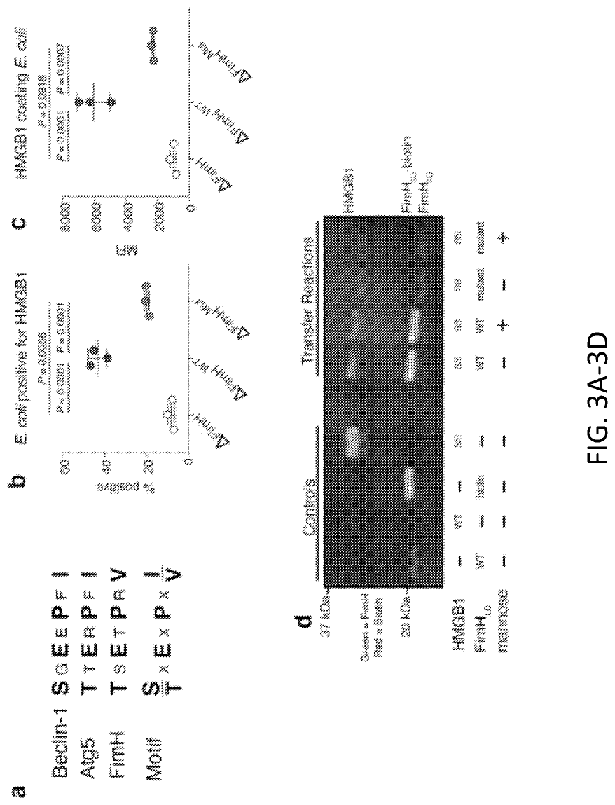

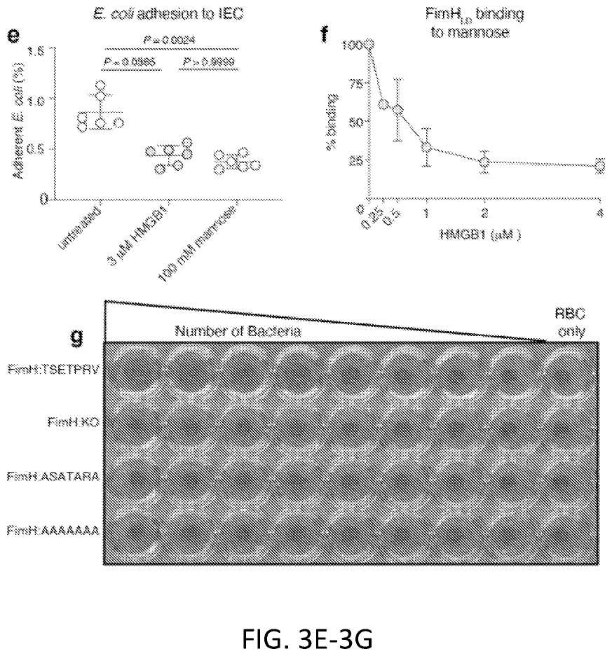

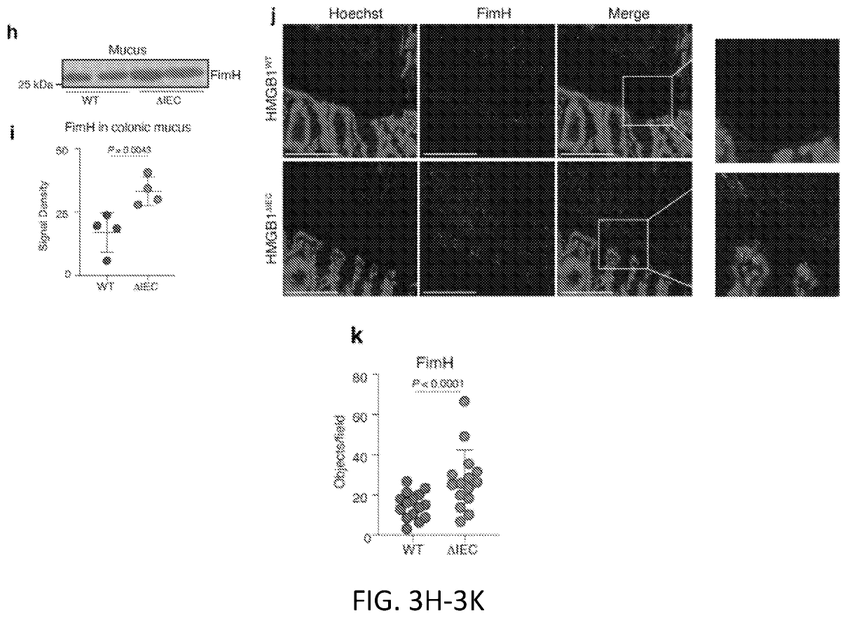

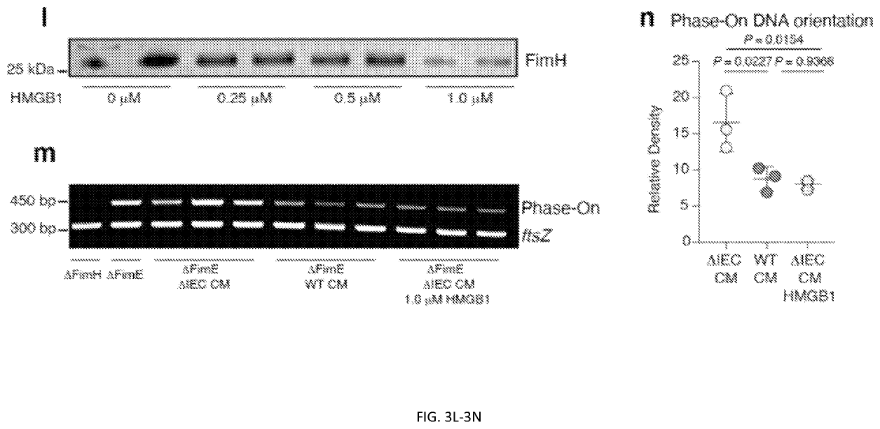

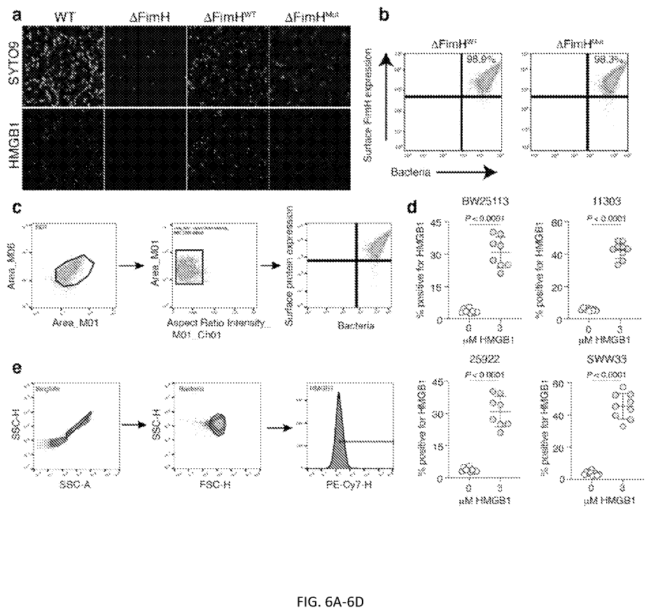

The observation that HMGB1 binds directly to gut microbes in vivo and in vitro led to the hypothesis that HMGB1 targets one or more proteins expressed on the surface of bacteria. In previous work, an amino acid sequence shared between the mammalian proteins Beclin-1 and Autophagy protein 5 (Atg5) that was targeted by cytosolic HMGB1 during microbial stress was identified (Zhu/Messer, JCI, 2015) Querying the PROSITE database using a motif derived from this sequence yielded a large number of hits in known or predicted bacterial adhesins, including FimH, a component of type 1 fimbrial (T1F) adhesion and perhaps the best characterized bacterial adhesin (FIG. 3a). FimH is a phase-inducible virulence factor that is carried by Enterobacteriaceae, including E. coli, and has been implicated in infectious diarrheal diseases, urinary tract infections, extraintestinal infections, colorectal cancers, and inflammatory bowel diseases. Expression of FimH is low in E. coli when they are in a commensal state, but it is highly expressed by pathogenic E. coli strains or when commensal strains become virulent. HMGB1 interaction with the conserved amino acid motif target of HMGB1 (ToH1) was verified in FimH expressed by E. coli. Recombinant human HMGB1 (rHMGB1) bound to E. coli expressing WT FimH, but the numbers of bacteria positive for HMGB1 and the amount of HMGB1 bound to each individual bacterium were significantly lower when cells lacked FimH (ΔFimH) or expressed FimH mutated in ToH1 (ΔFimHMUT) (FIG. 3b, c, FIG. 6a-6d). A label transfer assay was next employed to interrogate direct protein-protein interaction between HMGB1 and the IEC binding lectin domain of FimH (FimHLD). Label transferred to WT rFimHLD, but not rFimHLD mutated in ToH1, demonstrating that HMGB1 binds to FimH through ToH1 (FIG. 3d). Inclusion of mannose, the ligand bound by FimH for T1F adhesion, did not alter the efficiency of label transfer from rHMGB1 to WT rFimH. This and published structural data showing that ToH1 is distinct from the mannose binding site in FimH indicate that HMGB1 does not compete with mannose for binding to FimH. Auto transfer of label from rHMGB1 to itself was also observed in the WT rFimHLD reactions, supporting that HMGB1 forms an oligomer, most likely a dimer, when it interacts with FimH. This is consistent with the ability of HMGB1 to bind to FimH on more than one bacterial cell and cause aggregation.

Whether HMGB1 could limit E. coli adhesion to host cells was next examined. rHMGB1 inhibited E. coli adhesion to IEC at a level that was equivalent to mannose and inhibited red blood cell (RBC) agglutination in a dose-dependent manner (FIG. 3e). rHMGB1 also inhibited binding between rFimHLD and mannose showing that reduction in adhesion occurs due to the direct interaction between HMGB1 and FimH (FIG. 3f). Mutation of ToH1 in FimH severely impaired RBC agglutination by E. coli, implying that this sequence is necessary for the adhesin function of this protein (FIG. 3g).

HMGB1 Regulates Expression of Type 1 Fimbrial Adherence Machinery in E. coli

In mice lacking IEC HMGB1, FimH protein levels were higher in colonic mucus and in tissue than WT littermates (FIG. 3h, i, j, k). This is consistent with data demonstrating overall increased numbers of bacteria at the epithelial surface in ΔIEC mice. However, the increase in FimH could be due to increased numbers of bacteria expressing the same amount of FimH, increased FimH expression on each bacterium without a change in the number of bacteria, or both. Differences in relative abundance of E. coli or Enterobacteriaceae between the mouse genotypes by 16s rRNA gene sequencing were not observed and in vitro assays did not identify an effect of HMGB1 on E. coli colony forming units. Therefore, it was considered that HMGB1 could regulate bacterial expression of FimH. Exposure to rHMGB1 decreased FimH expression by commensal E. coli in a dose-dependent manner. (FIG. 3l). In order to determine whether HMGB1 causes E. coli to turn off FimH expression or prevents bacteria from turning it on, a FimE knockout (ΔFimE) strain of E. coli was used. The genes required for type 1 fimbriae are arranged in a single operon that is regulated by a DNA switch region. The switch between fimbrial producing (FimON) and non-producing (FimOFF) states is regulated by two recombinases, FimB and FimE. When FimE is knocked out, bacteria can switch to FimON, but switching to FimOFF is impaired, providing a relative “counter” for the switch to FimON. Media conditioned by WT primary intestinal epithelial cells repressed FimON in comparison to media conditioned by cells derived from ΔIEC mice (FIG. 3m, n). Furthermore, addition of HMGB1 to the ΔIEC conditioned media repressed FimON similarly to WT media. Thus, these combined results indicate that HMGB1 in the environment binds to its evolutionarily conserved target sequence (ToH1) in FimH, inhibits FimH binding to its target ligand mannose, impairs T1F adhesion to host cells, and suppresses expression of this adhesion mechanism.

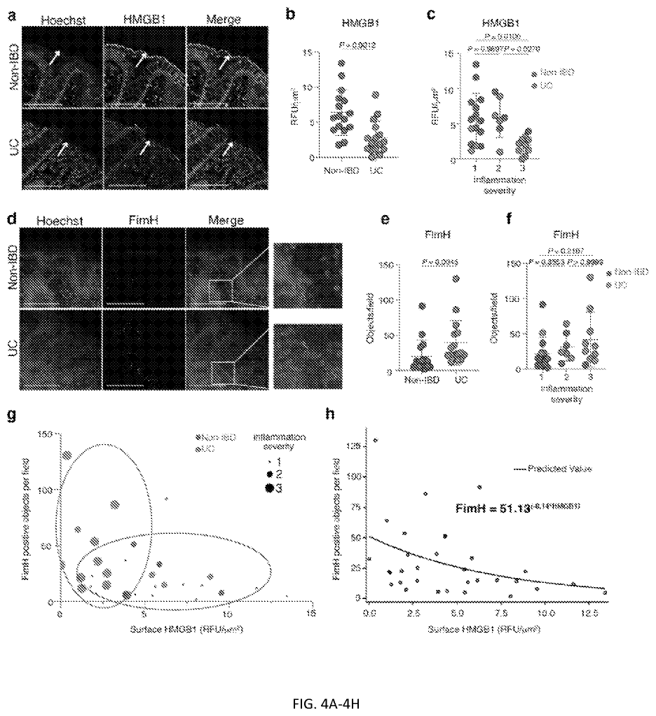

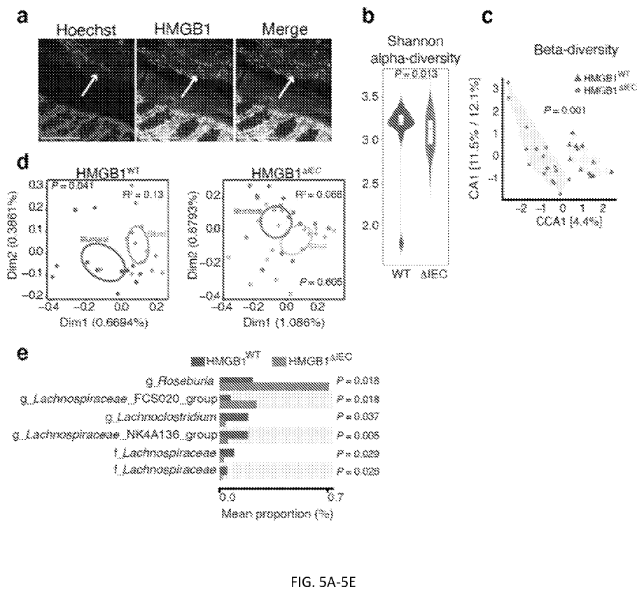

It was next investigated whether HMGB1 mucosal defense released from IEC is compromised in IBD patients. There are two major subtypes of IBD, Crohn's disease and ulcerative colitis. Ulcerative colitis affects only the colon and is thought to result from tissue damage initiated at the luminal surface of the epithelium. Surface-associated HMGB1 was generally easily appreciable in resected colon tissue from non-IBD patients, whereas tissue from UC patients commonly had very low levels of HMGB1 with a patchy appearance (FIG. 4a, b and Table 2.). Surface associated HMGB1 was lowest in patients with severe inflammation and was absent in areas devoid of IEC (FIG. 4c). FimH was assayed in serial tissue sections from the same patients and higher numbers of FimH positive bacteria in tissue from UC patients were found (FIG. 4d, e) However, FimH was not related to inflammation severity (FIG. 4f). When surface HMGB1 and FimH were plotted from the same patient, UC patients clustered together with low HMGB1 and high FimH, while non-IBD patients clustered together with higher HMGB1 and low FimH (FIG. 4g). Mathematically modeling the relationship between HMGB1 and FimH demonstrated that the number of FimH positive bacteria in tissue was dependent on HMGB1 with levels of FimH increasing as HMGB1 decreased (FIG. 4h). Thus, UC is characterized by failure of HMGB1 defense with a concomitant increase in tissue-associated bacteria expressing the HMGB1 target protein FimH.

TABLE 2
Patient Demographics

| Demographics | Non-IBD | UC |
|---|---|---|
| Total | 16 | 16 |
| Age, mean, (SD), [range] | 52.9 (±13.2) [34-75] | 40 (±15.5) [22-76] |
| Age in males | 56.3 (±13.6) [36-75] | 41.8 (±15.2) [23-69] |
| Age in females | 47.2 (±47.2) [34-59] | 38.3 (±16.7) [22-76] |

Sex, n (%)

| | | | | |
|---|---|---|---|---|
| Male | 10 | (62.5%) | 8 | (50%) |
| Female | 6 | (37.5%) | 8 | (50%) |

Disease, n (%)

| | | | |
|---|---|---|---|
| Adhesions | 1 | (6.25%) | — |
| Malrotation | 1 | (6.25%) | — |
| Constipation | 2 | (12.5%) | — |
| Diverticulitis | 9 | (56.3%) | — |
| Cancer | 3 | (18.8%) | — |

Inflammation severity

| | | | | |
|---|---|---|---|---|
| 1 (none/mild), n (%) | 12 | (75.0%) | 4 | (25.0%) |
| 2 (moderate), n (%) | 3 | (18.8%) | 5 | (31.3%) |
| 3 (severe), n (%) | 1 | (6.25%) | 7 | (43.8%) |

Ethnicity, n (%)

| | | | | |
|---|---|---|---|---|
| White | 15 | (93.8%) | 15 | (93.8%) |

| | | | |
|---|---|---|---|
| Black | 1 | (6.25%) | 0 |

| | | | |
|---|---|---|---|
| Unknown | 0 | 1 | (6.25%) |

High mobility group box 1 (HMGB1) is an abundant, ubiquitously expressed protein that has intra- and extracellular functions in many different cell types. Here it is shown that HMGB1 is an active component of front-line mucosal barrier defense in the colon with direct and indirect effects to limit virulence of the gut microbiota. The cross-kingdom protein-protein interaction between mammalian HMGB1 and bacterial FimH directly limits bacterial virulence by inactivating adhesion through FimH. Expression of T1F genes is also suppressed by HMGB1, suggesting that bacteria like E. coli regulate phase or virulence in response to HMGB1. The relationship between low levels of HMGB1 and high levels of FimH positive bacteria was noted in both a murine model and in resected colonic tissue from ulcerative colitis patients in the studies herein.

Ulcerative colitis has long been linked to adherent microbes, but no single pathogen has been consistently identified across studies. Herein it is suggested that HMGB1, a component of host defense, fails in UC patients, allowing bacteria to switch on adhesion mechanisms normally suppressed by HMGB1 and adhere to host tissues. This data provide an explanation for why E. coli and potentially other bacteria adhere to intestinal tissue in UC. The molecular target of HMGB1 in mucosal host defense is a small, evolutionarily conserved amino acid sequence found in adhesins from many different types of bacteria. Much like the ligands that activate pattern recognition receptors, ToH1 appears to be broadly utilized, critical for virulence, and difficult to modify without losing function. E. coli FimH was used as the exemplar ToH1 positive protein in mechanistic studies herein since FimH and T1F adhesion are well characterized. The ToH1 sequence is identical and present in FimH from all E. coli genomes examined, including E. coli that cause infectious diarrheas, strains associated with chronic urinary tract infection, extraintestinal pathogenic E. coli, and IBD-associated adherent and invasive E. coli. The discovery of ToH1 in FimH has important therapeutic implications, as T1F adhesion is a preferred system for E. coli and preventing T1F adhesion thus provides a novel therapeutic strategy for diseases caused by this organism. Bacterial adhesion mechanisms are high value therapeutic targets for nearly all bacterial diseases since blocking adhesion prevents tissue damage, inflammation, and immune activation in disease models and patients.

### Example 2

Adhesion is the first step of microbial disease and blocking adhesion has the potential to prevent infection. HMGB1 binding to ToH1 prevents microbial adherence proteins from binding to their carbohydrate target on mammalian host cells to anchor the bacteria in place. Failure of this defense is likely relevant for multiple diseases where E. coli adheres to the gut epithelium, including infectious diarrheas, colorectal cancer, and IBD. Moreover, the ToH1 sequence and HMGB1 inhibition of adhesion functions are not restricted to a specific type of microbe and thus has potential applications for bacteria, viruses, fungi, and protozoa. Accordingly, ToH1 provides a novel molecular target for diagnosis and treatment of disease.

ToH1 is a 7 aa sequence present in surface-expressed adhesins from bacteria, virus, fungi, and protozoa. The sequence is also present in mammalian proteins, most of which are intracellular. Accordingly, therapeutically targeting ToH1 may also have anti-inflammatory function. Exemplary nonlimiting organisms/pathogens with adhesins that contain ToH1 are shown in Table 3.

| Type of Microorganism | Name of Microorganism | Disease | Morbidity* | Mortality\hat{ } |
|---|---|---|---|---|
| Bacterium (Gm−) | Escherichia coli | Diarrhea, UTI, IBD | 300,000,000 | 200,000 |
| Bacterium (Gm+) | Staphylococcus aureus | Staph infections, MRSA | 390,000 | 18,000 |
| Bacterium (Spirochete) | Borrelia burgdorferi | Lyme disease | 300,000 | Uncommon |
| Bacterium (Mycobacterium) | Mycobacterium tuberculosis | Tuberculosis | 10,000,000 | 1,300,000 |
| Fungus (Yeast) | Candida albicans | Invasive Candidiasis | 700,000 | 280,000 |
| Virus (dsDNA) | Human herpesvirus 4 | Mononucleosis, lymphoma | 6,750,000,000 | 143,000 |
| Virus (−ssRNA) | Influenza B virus | Seasonal flu | 5,000,000 | 650,000 |
| Virus (+ssRNA) | Dengue Virus | Dengue | 390,000,000 | Uncommon |
| Apicomplexon (Blood | Plasmodium vivax | Malaria | 219,000,000 | 435,000 |
| parasite) | | | | |
| Diplomonadiad (Intestinal | Giardia intestinalis | Infectious diarrhea | 200,000,000 | Rare |
| parasite) | | | | |
| Amcebozoan (Amoeba) | Entamoeba histolytica | Infectious diarrhea | 500,000,000 | 100,000 |
| Trypanasomid (Systemic | Trypanosoma cruzi | Chagas disease | 8,000,000 | 10,000 |
| parasite) | | | | |

*Estimated numbers of humans infected and the number that die from the infecious disease each year, worldwide.

Provided herein are antibodies that mimic the function of HMGB1. The antibodies block adhesion and are useful for treatment or prevention of microbial disease, including both infectious diseases and chronic inflammatory diseases caused by microbes. Antibodies provided herein are further shown to bind to ToH1 in the mammalian IL-1 receptor 1 (IL1R1) and block pro-inflammatory signaling (e.g. inhibit signaling through IL1R). The antibodies provided herein can also be used to diagnose microbial disease since the adhesins that contain the ToH1 sequence are usually not expressed or expressed only at low levels in commensal microbes and would not be expected to be associated with tissues in the healthy state.

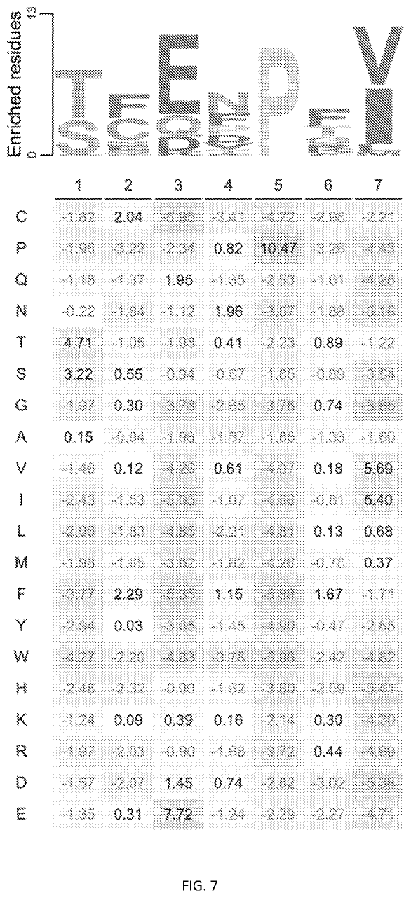

Experiments were conducted to determine the HMGB1 target sequence. A set of sequences and ToH1 positive proteins were verified for HMGB1 inhibition of binding to target carbohydrate in a plate assay. A position specific scoring matrix (http://slim.icr.ac.uk/pssmsearch/) was used to generate a bioinformativc prediction of the HMGB1 target sequence based upon experimental data. Results are shown in FIG. 7. Numbers shown in the figure reflect the likelihood of the given residue being in that position. Analysis of this sequence in publicly available protein crystal structures suggests that the sequence determines a surface exposed structure, and that similarities of sequence seem to predict similarities of structure.

Antibodies targeting ToH1 in FimH were obtained through a phage display method. A human phage display library was utilized to identify ToH1 binding scFvs. The proprietary Proteogenix human naïve library was used (https://us.proteogenix.science/antibody-production/phage-display-services/#). 368 human donors from 5 different ethnic groups were used for maximized diversity, resulting in 5.37×10¹⁰ scFv and Fabs. The library was first screened against the ToH peptide from E. coli FimH (TSETPRV (SEQ ID NO: 33)). Hits from the initial screen were then screened against the functional E. coli FimH lectin domain protein.

Three lead scFvs were identified and sequenced. The sequences are provided below.

Antibody Sequences:
>F5(259 AAs*, 27.51 kDa*, pI: 9.18*)
(SEQ ID NO: 7)
MGYLLPTAAAGLLLLAAQPAMAEVQLVQSGPEVKKPGASVKVSCKASGFIFSNYGI
GWVRQAPGQGLEWLGWISGYNGQTNYAQTVQGRVTMTADTSTTTAYMDLRNLRSDD
TAIYYCARQSIPYYMDVWGKGTMVTVSSGGGGSGGGGSGGGGSGGGGASDIVMTQS
PLSSPVTLGQPASISCRSSQSLVHSNGNTYLSWLHQRPGQPPRLLIYRISNRLSGV
PDRFSGSGAGTDFTLKISRVEAEDVGVYYCMQAKQFPVTFGQGTRLEIKGSHHHHH
H
Features: Signal peptide: [1:22] F5-scFv: [23:273]
scFv linker: [141:161]
6His with linker: [274:281]
*Theoretical values calculated without signal peptide
F5, VH, putative CDRs shown in bold
(SEQ ID NO: 1)
EVQLVQSGPEVKKPGASVKVSCKASGFIFSNYGIGWVRQAPGQGLEWLGWISGYN
GQTNYAQTVQGRVTMTADTSTTTAYMDLRNLRSDDTAIYYCARQSIPYYMDVWG
KGTMVTVSS
F5, VL, putative CDRs shown in bold-
(SEQ ID NO: 2)
DIVMTQSPLSSPVTLGQPASISCRSSQSLVHSNGNTYLSWLHQRPGQPPRLLIYRISN
RLSGVPDRFSGSGAGTDFTLKISRVEAEDVGVYYCMQAKQFPVTFGQGTRLEIK
>F11(261 AAs*, 27.25 kDa*, pI: 9.21*)
(SEQ ID NO: 8)
MGYLLPTAAAGLLLLAAQPAMAQVQLVQSGAEVKKPGASVKVSCKASGYTFTGYYM
HWVRQAPGQGLEWMGWINPNSGGTNYAQKFQGRVTMTRDTSISTAYMELSRLRSD
DTAVYYCARDRGSGATRYGMDVWGQGTLVTVSSGGGGSGGGGSGGGGSGGGGASQ
SALTQPASVSGSPGQSITISCTGTSSDIGNYNYVSWYQQHPGKAPKLMIYDVTKR
PSGVSNRLSGSKSGNTASLTISGLQAEDEADYYCSSYTGRSSWVFGGGTKLTVLG
SHHHHHH
Features: Signal peptide: [1:22] F11-scFv: [23:275]
scFv linker: [145:165]
6His with linker: [276:283]
*Theoretical values calculated without signal peptide F11
F11, VH, putative CDRs shown in bold-
(SEQ ID NO: 3)
QVQLVQSGAEVKKPGASVKVSCKASGYTFTGYYMHWVRQAPGQGLEWMGWINPN
SGGTNYAQKFQGRVTMTRDTSISTAYMELSRLRSDDTAVYYCARDRGSGATRYG
MDVWGQGTLVTVSS
F11, VL, putative CDRS shown in bold-
(SEQ ID NO: 4)
QSALTQPASVSGSPGQSITISCTGTSSDIGNYNYVSWYQQHPGKAPKLMIYDVTK
RPSGVSNRLSGSKSGNTASLTISGLQAEDEADYYCSSYTGRSSWVFGGGTKLTVL
>G6(263 AAs*, 28.25 kDa*, pI: 8.80*)
(SEQ ID NO: 9)
MGYLLPTAAAGLLLLAAQPAMAQVQLQQSGPGLVKPSQTLSLTCAISGDSVSSNSAAW
NWIRQSPSRGLEWLGRTYYRSKWYNDYAVSVKSRITINPDTSKNQFSLQLNSVTPED
TAVYYCARRSTWGTFDYWGQGTLVTVSSGGGGSGGGGSGGGGSGGGGASDIVMT
QTPDSLAVSLGERATINCKSSQSVLYSSNNKNYLAWYQQKPGQPPKLLIYWASTRES
GVPDRFSGSGSGTDFTLTISSLQAEDVAVYYCQQYYALPLTFGGGTKLEIKGSHHHH
HH
Features: Signal peptide: [1:22] G6-scFv: [23:277]
scFv linker: [144:164]
6His with linker: [278:285]
*Theoretical values calculated without signal peptide
G6, VH, putative CDRS shown in bold-
(SEQ ID NO: 5)
QVQLQQSGPGLVKPSQTLSLTCAISGDSVSSNSAAWNWIRQSPSRGLEWLGRTYYRS
KWYNDYAVSVKSRITINPDTSKNQFSLQLNSVTPEDTAVYYCARRSTWGTFDYWG
QGTLVTVSS
G6, VL, putative CDRS shown in bold-
(SEQ ID NO: 6)
DIVMTQTPDSLAVSLGERATINCKSSQSVLYSSNNKNYLAWYQQKPGQPPKLLIYW
ASTRESGVPDRFSGSGSGTDFTLTISSLQAEDVAVYYCQQYYALPLTFGGGTKLEIK

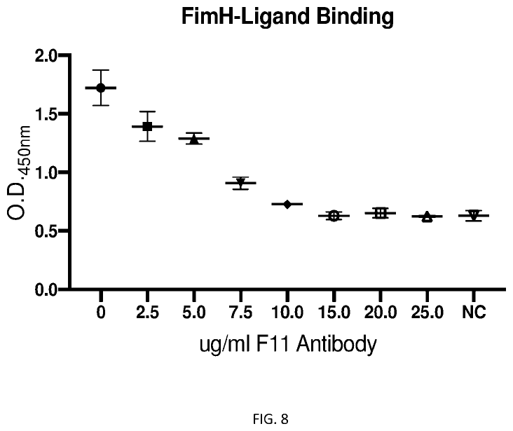

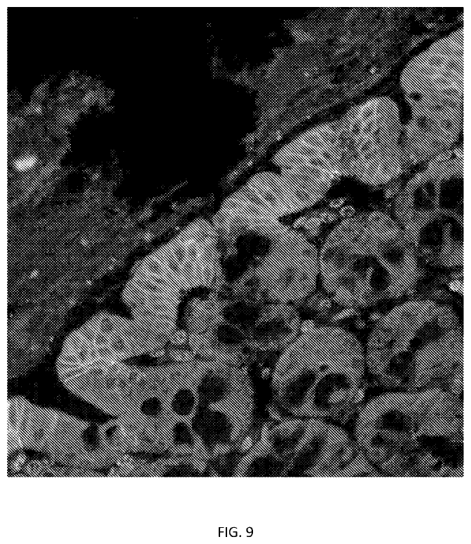

scFvs were chemically synthesized with optimization for expression in E. coli (signal peptide for periplasmic targeting at the N-term and 6His-Tag at the C-term). Antibody characterization indicates that antibody F11 recognizes E. coli FimH at the ToH site and has blocking activity in initial in vitro studies. Exemplary results showing inhibition of FimH binding to the target ligand, mannose, is shown in FIG. 8. Antibody F11 was incubated with FimH and then FimH binding to mannose was measured in an ELISA format. NC is negative control (detection reagent only). F11 inhibited FimH binding to mannose in a dose-dependent manner. Moreover, antibody characterization shows that anti-ToH1 antibodies are suitable for diagnostic purposes, as shown by successful immunofluorescence labeling of colonic tissue from an HMGB1 deficient mouse using the exemplary antibody F11. Results demonstrate that the antibody is suitable for immunostaining to reveal ToH1 positive microbes in close proximity or attached to the intestinal surface.

Table 4 shows F5, F11, and G6 binding to E. coli FimH. Phages displaying sequences for F5, F11, and G6 were used. FimH protein was unloaded or loaded with mannose. The F5, G6, and F11 sequences were all shown to recognize E. coli FimH.

TABLE 4

| | Coating | | | | | |
|---|---|---|---|---|---|---|
| | | | Pur. FimH | | PBS (negative | |
| Clone ID | Pur. FimH unloaded | | Mannose-loaded | | control) | |
| F5 | 0.72 | 0.77 | 0.58 | 0.62 | 0.02 | 0.03 |
| G6 | 0.4 | 0.35 | 0.29 | 0.36 | 0.05 | 0.03 |
| F11 | 0.17 | 0.19 | 0.02 | 0.04 | 0.03 | 0.02 |

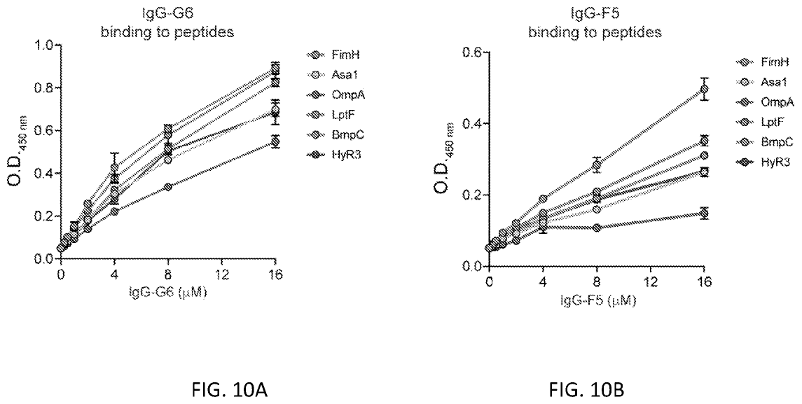

Antibody binding was further verified by ELISA. Results are shown in FIG. 10A (G6) and FIG. 10B (F5). ToH1 peptide sequences represented are: FimH (E. coli, TSETPRV (SEQ ID NO: 33), Asa1 (Enterococcus faecalis, TKENPFV (SEQ ID NO: 39)), OmpA (Bacteroidales, SFELPTI (SEQ ID NO: 35)), LptF (E. coli, SPEKPTV (SEQ ID NO: 36), BmpC (Borrelia burgdorferi, SYERPDI (SEQ ID NO: 40), and HyR3 (Candida albicans, TFEPPVV (SEQ ID NO: 38).

### Example 3

Antibodies broadly targeting ToH1 were obtained through a serial peptide injection method. Antigen design and immunization were developed for broader reactivity against a short/universal target sequence. The goal of the immunization strategy is to generate reactivity against only the “fixed” amino acids in the motif (named amino acids). Two strategies were employed.

Strategy 1: Sequential immunization with 4 peptides that each vary by one of the “fixed amino acids”. For this strategy, peptides that follow a motif shown in the table below were used, wherein “x” denotes a variable amino acid and fixed amino acids are identified by specific amino acids that are suitable. The motifs identified in the table below follow the motif [S/T]xExPx[I/V], where “x” denotes a variable amino acid.

| Motif (this is the | |
|---|---|
| pattern, but not | Actual peptide used |
| necessarily the | (these are examples |
| order of peptides | of the specific types |
| that should be | of peptides that |
| used) | should be used) |
| TxExPxV | TSETPRV (SEQ ID NO: 33) |
| SxExPxI | SSERPMI (SEQ ID NO: 34) |
| SxExPxV | SPEKPTV (SEQ ID NO: 36) |
| TxExPxI | TRELPQI (SEQ ID NO: 37) |

The antibody produced through this method was shown to have FimH blocking activity in initial studies. This was a polyclonal rabbit antibody. Monoclonal/recombinant antibodies with blocking activity may also be developed using this strategy.

Strategy 2: A sequential vaccination strategy was designed using a combination of the experimentally confirmed ToH1 (Target of HMGB1) epitopes together with epitopes with enriched residues designed based on a combination of the bioinformatic analysis output, comparison to the naturally identified sequences, and optimization of residue charged aimed at for a hypothesized maximization of binding of the resulting antibody response.

Peptides following the motif [S/T]xExPx[I/V] were used, with specific amino acids tested at each variable peptide position (each “x’).

Peptides having a formula identified below can be used for vaccination.

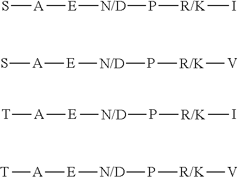

The first residue is a fixed residue, S or T.

The second residue is a variable amino acid. For some peptides, the second residue was designed to be A, which allows “killing” 2^{nd} residue to make sure it is not involved in binding. A is neutral and won't impact binding.

The third residue is a fixed residue, E.

The fourth residue is a variable amino acid. For some peptides, the fourth residue was designed to be either N or D. Either N or D are acceptable residues, and add similar hydrophilicity and have good prevalence based on the sequence logo. This 4^{th} residue cannot be “killed’ into A, as it would make the peptide too hydrophobic.

The fifth residue is a fixed residue, P.

The sixth residue is a variable amino acid. For some peptides, the sixth residue was designed to be either R or K. Either R or K are acceptable and add similar hydrophilicity and have some prevalence based on the sequence logo. This 6^{th} residue cannot be killed into A as would make peptide too hydrophobic.

The seventh residue is a fixed residue, I or V.

All combinations were predicted to be very similar in terms of suitability and chances of success.

Specific peptide sequences used for injection were as follows:

### Injection #

1
(SEQ ID NO: 33)
TSETPRV
2
(SEQ ID NO: 42)
SAENPKI
3
(SEQ ID NO: 41)
TAEDPRI
4
(SEQ ID NO: 36)
SPEKPTV

Following injection, antibodies were isolated from the host and tested for binding to ToH1 peptides and ToH1 positive proteins.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

### Example 4

### Nanobodies

Nanobodies were derived from animals immunized with the novel strategy for producing anti-ToH1 antibodies (e.g. described in Example 3). After immunization, lymphocytes were collected and used to construct a phage display library of antibody sequences. The library was screened for ToH1 peptide binders and then hits were re-screened for binding to ToH1 positive proteins.

The cDNAs of 2 VHHs identified via phage display were chemically synthesized with optimization for mammalian expression in CHO cells and Avi-tag-6His-tag at C-terminus, then sub-cloned into mammalian cell expression vector pTXs1. Expected corresponding proteins produced are illustrated below. The VHH were then again tested for binding to ToH1 positive proteins.

>G2-VHH 164 aa (also referred to as VHH-G2)
(SEQ ID NO: 31)
MKHLWFFLLLVAAPRWVLSQVQLVESGGGLVQPGGSLRLSCAASGFTMGDYAIGWFR
QVPGKEREGLSYSTKNGIKAYADSVRDRFTISRENAKNTVYLQMNSLKPEDTAVYY
CAASTRFGGLLVSRYDYWGQGTQVTVSSGLNDIFEAQKIEWHEGSHHHHHH
Features:
Signal peptide: [1:19]
G2-VHH: [20:141]
Avi-tag: [142:156]
6His-tag with linker: [157:164]
>F7-VHH 164 aa (also referred to as VHH-F7)
(SEQ ID NO: 32)
MKHLWFFLLLVAAPRWVLSQLQLVESGGGLVQAGGSLRLSCAASGLTVSSYSMGWFR
QAPGKEREFVAAISRSGATINYGSSVQGRFMIARDDAKNTVNLQMNSLKPEDTAVY
YCAARDRYSLVARAYEYWGQGTQVTVSSGLNDIFEAQKIEWHEGSHHHHHH
Features:
Signal peptide: [1:19]
F7-VHH: [20:141]
Avi-tag: [142:156]
6His-tag with linker: [157:164]

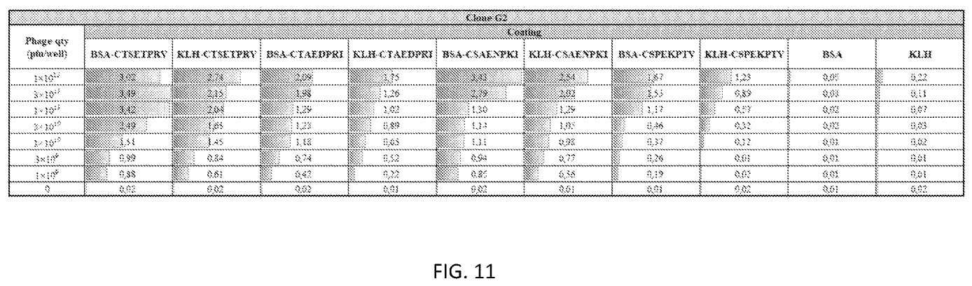

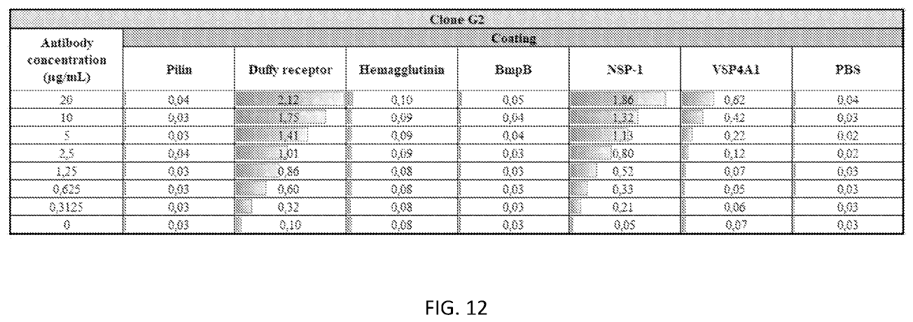

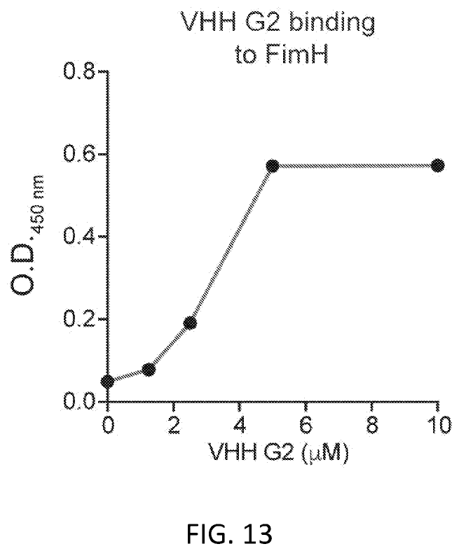

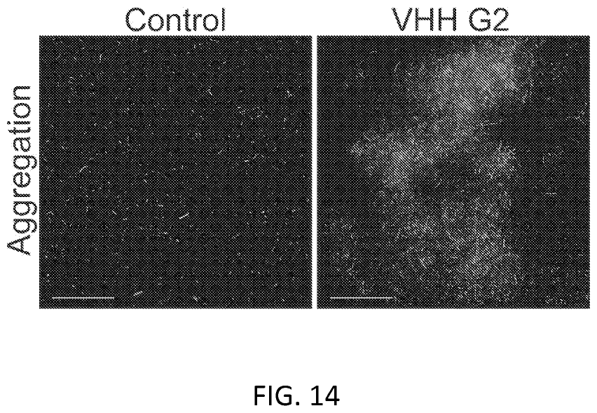

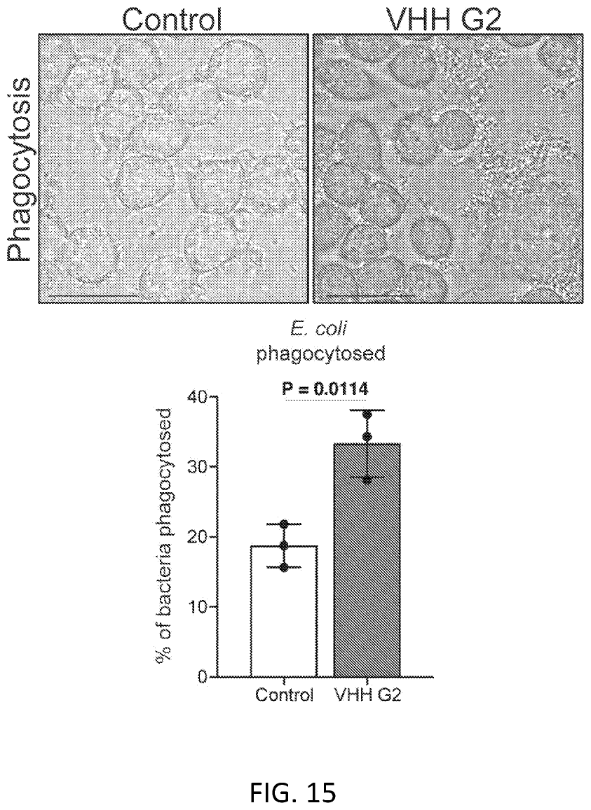

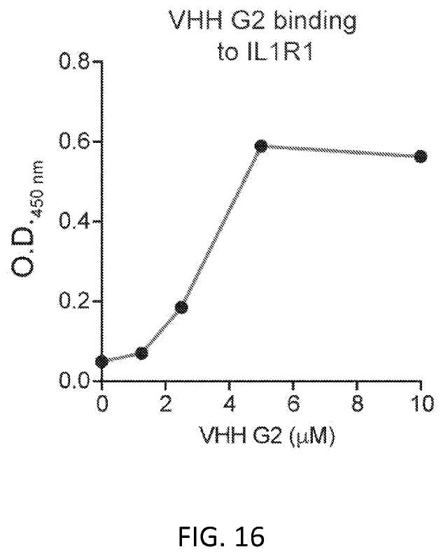

Binding activity of VHH-G2 was evaluated. Results are presented in FIG. 11-FIG. 16. VHH-G2 was shown to bind to all ToH1 peptides (FIG. 11) and to bind to ToH1 positive adhesins (FIG. 12). VHH-G2 was also shown to bind to E. coli FimH (FIG. 13) and to bind to and aggregate E. coli (FIG. 14). VHH-G2 was also shown to increase bacterial clearance by macrophages (FIG. 15). VHH-G2 was also shown to bind to human IL1R1 (FIG. 16), suggesting that VHH-G2 binds to IL1R1 and inhibits and inhibits pro-inflammatory signaling.

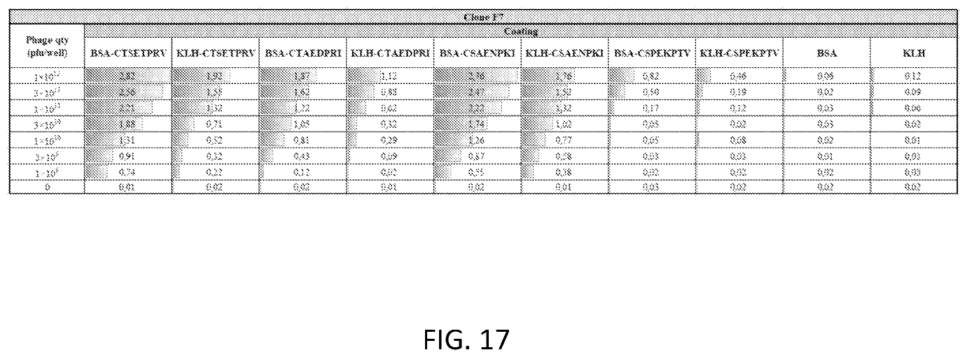

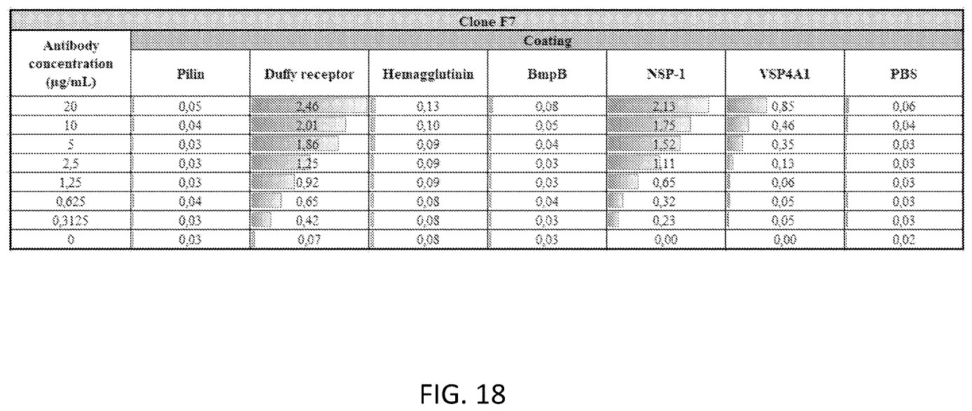

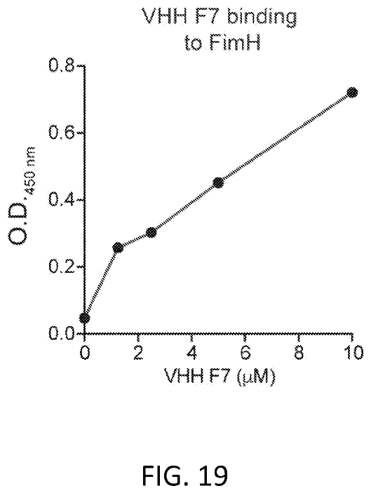

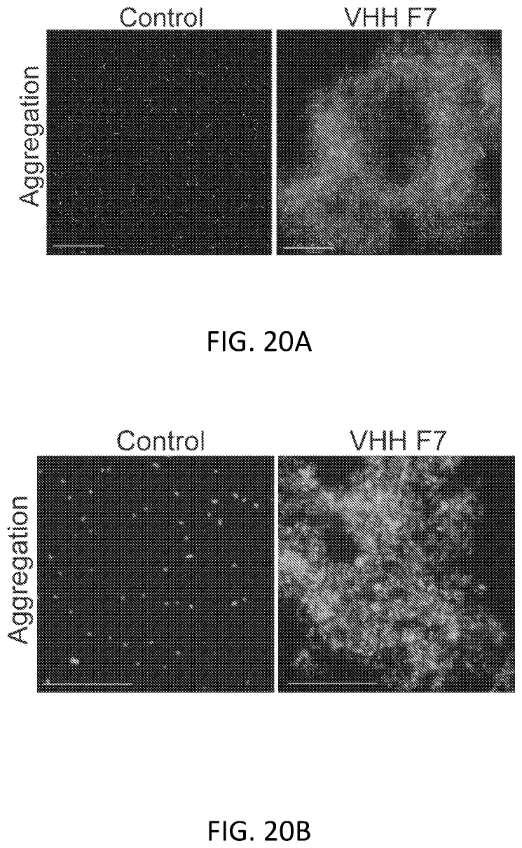

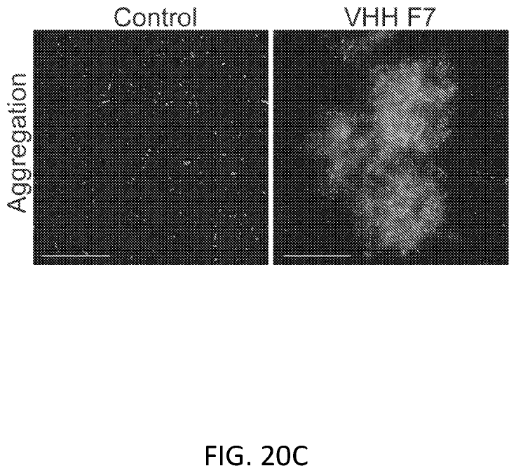

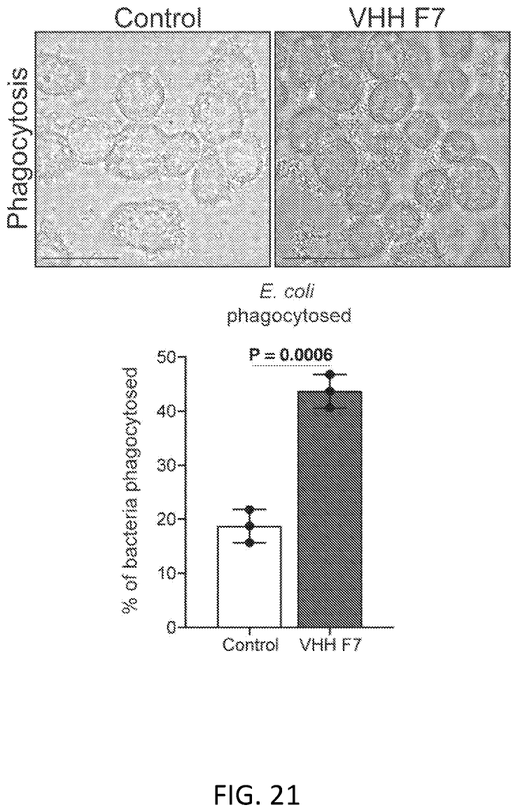

Binding activity of VHH-F7 was evaluated. Results are presented in FIG. 16-FIG. 22. VHH-F7 was shown to bind to ToH1 peptides (FIG. 17) and to bind to ToH1 positive adhesins (FIG. 18). VHH-F7 was also shown to bind to E. coli FiMH (FIG. 19). VHH-F7 was also shown to bind to and aggregate multiple bacterial species, including E. coli (FIG. 20A), S. aureus (FIG. 20B), and bacteria from a complex community (FIG. 20C). VHH-F7 was also shown to increase bacterial clearance by macrophages (FIG. 21).

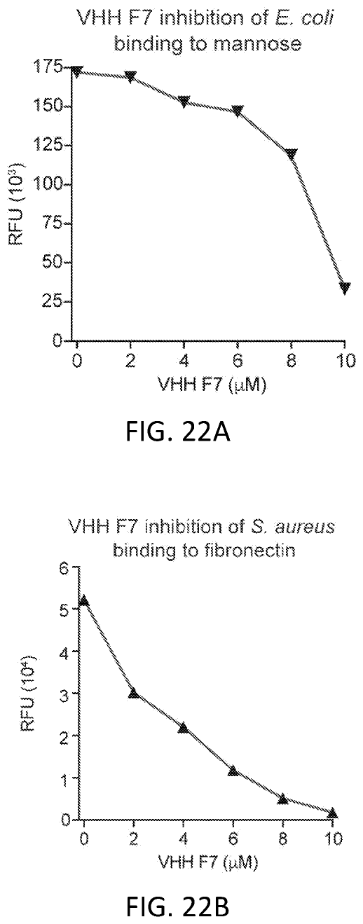

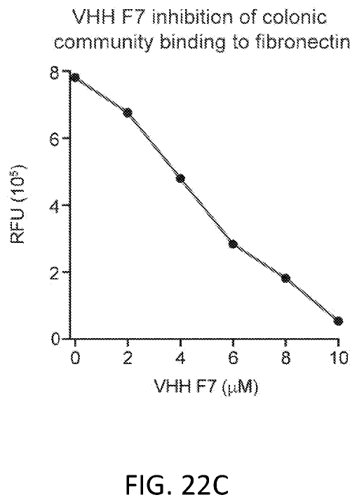

Adhesion inhibition assays were performed which demonstrate that VHH-F7 inhibits E. coli binding to mannose (FIG. 22A), S. aureus binding to fibronectin (FIG. 22B), and bacterial community binding to fibronectin (FIG. 22C).

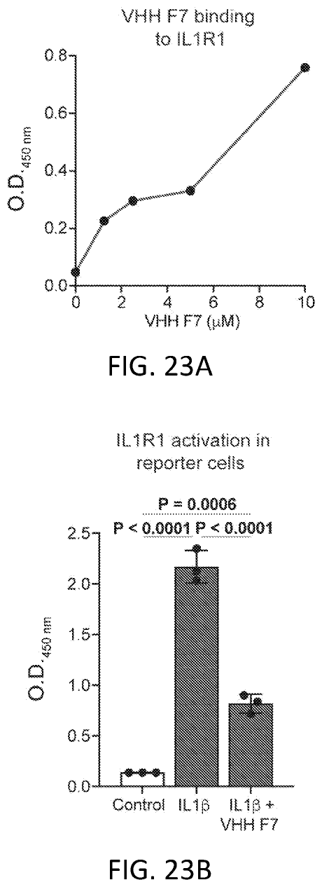

VHH-F7 was shown to bind to IL1R1 (a mammalian ToH1 positive protein) by ELISA (FIG. 23A), and to inhibit IL1R signaling (FIG. 23B).

### Methods

E. coli Aggregation Assay

SWW33-GFP E. coli were grown overnight at 37 degrees C. in nutrient broth. After overnight growth, the culture was diluted back to an OD of 0.8. 200 ul of the OD 0.8 culture was added to fresh Eppendorf tubes. The bacteria were pelleted at 5000 RPM for 5 minutes and then resuspended in 100 ul of TBS with 10 mM CaCl2. This 100 ul was then split in half with 50 ul used for control and the other 50 ul used for VHH F7 treatment. VHH F7 was added to the 50 ul reaction to a final concentration of 10 uM. Once the treatment was added, the reactions were placed at 37 degrees C. for 2 hours to aggregate. Then, 3 ul of reaction was placed onto a rectangular glass coverslip and sandwiched with a 0.15% agarose pad and imaged on a fluorescence microscope.

S. aureus Aggregation Assay

Staphylococcus aureus was grown overnight at 37 degrees C. in BHI broth. After overnight growth, the culture was diluted back to an OD of 0.8. Biotium Cf™ Dye Lectin Conjugates Wheat germ agglutinin CF™488A was added to the culture and allowed to incubate for 30 minutes at 37 degrees C. to allow for the fluorescent labeling of the bacteria. 200 ul of the OD 0.8 culture was added to fresh Eppendorf tubes. The bacteria were pelleted at 5000 RPM for 5 minutes and then resuspended in 100 ul of TBS with 10 mM CaCl2. This 100 ul was then split in half with 50 ul used for control and the other 50 ul used for VHH F7 treatment. VHH F7 was added to the 50 ul reaction to a final concentration of 10 uM. Once the treatment was added, the reactions were placed at 37 degrees C. for 2 hours to aggregate. Then, 3 ul of reaction was placed onto a rectangular glass coverslip and sandwiched with a 0.15% agarose pad and imaged on a fluorescence microscope.

### B6 Microbiota Colonic Community Aggregation Assay

Colons from SPF C57BL.6J mice were excised from a freshly euthanized mouse between the ages of 8-12 weeks old and fileted open longitudinally. An inoculating loop was used to scrape the contents into a 1.5 mL Eppendorf containing 1 mL of PBS. Contents were centrifuged at 400×G for 5 minutes; the supernatant was strained using a 70 um cell strainer. The flow through was washed twice with PBS and once with TBS containing 10 mM CaCl2 at 10,000×G for 2 minutes. After the third wash the sample was resuspended in 500 uL TBS 10 mM CaCl2 and Cell-Brite 488 Membrane Stain and allowed to incubate for 30 minutes at 37 degrees C. to allow for fluorescent labeling of the bacteria. The sample was split into 100 ul aliquots and centrifuged at 10,000×G for 2 minutes. The final washed and labeled pellet of B6 microbiota was resuspended in 100 ul of TBS with 10 mM CaCl2. This 100 ul was then split in half with 50 ul used for a control and the other 50 ul used for VHH F7 treatment. VHH F7 was added to the 50 ul reaction to a final concentration of 10 uM. Once the treatment was added, the reactions were placed at 37 degrees C. for 2 hours to aggregate. Then, 3 ul of reaction was placed onto a rectangular glass coverslip and sandwiched with a 0.15% agarose pad and imaged on a fluorescence microscope.

E. coli Phagocytosis Assay

Wild-type and HMGB1 KO RAW264.7 macrophages were plated at 250,000 cells/mL onto poly-L-lysine coated glass bottom dishes and allowed to incubate overnight at 37 degrees C. 5% CO₂ in DMEM+10% FBS. SWW33-GFP E. coli were grown overnight at 37 degrees C. in nutrient broth. The following day, the macrophages were inoculated to a final OD of 0.8 with SWW33-GFP E. coli (Bacterial input: 64×10\hat{ }8 cells) and treated with 10 uM VHH F7/G2 or control. The treated bacteria were then incubated with the macrophages for 1 hour at 37 degrees C. and then were imaged under a brightfield microscope. Phagocytosis can be observed at the surface of the bacteria where the bacteria appear to be captured and in the process of being engulfed.

Inhibition of E. coli Binding to Mannose (Adhesion Inhibition Assay)

SWW33-GFP E. coli were grown overnight at 37 degrees C. in nutrient broth. After overnight growth, the culture was diluted back to an OD of 0.8. 200 ul of the OD 0.8 culture was added to fresh Eppendorf tubes. The bacteria were treated with a dilution series of VHH F7 (0-10 uM) and incubated for 2 hours at 37 degrees C. Following incubation, the treated bacteria were carefully plated onto a mannose coated plate in triplicate, sealed and incubated at 37 degrees C. for 1 hour. After the 1-hour incubation, the bacteria were decanted from the plate and the plate was gently rinsed twice with 200 ul/well of PBS. Following the final wash step, the plate was read in a spectrophotometer at 485/538 (Ex/Em) to measure the number of bacteria bound to the mannose.

Inhibition of S. aureus Binding to Fibronectin (Adhesion Inhibition Assay)

S. aureus was grown overnight at 37 degrees C. in BHI broth. After overnight growth, the culture was diluted back to an OD of 0.8 and labeled for 30 minutes at 37 degrees C. with Biotium Cf™ Dye Lectin Conjugates Wheat germ agglutinin CF™488A. 200 ul of the OD 0.8 labeled culture was added to fresh Eppendorf tubes. The bacteria were treated with a dilution series of VHH F7 (0-10 uM) and incubated for 2 hours at 37 degrees C. Following incubation, the treated bacteria were carefully plated onto a fibronectin coated plate in triplicate, sealed and incubated at 37 degrees C. for 1 hour. After the 1-hour incubation, the bacteria were decanted from the plate and the plate was gently rinsed twice with 200 ul/well of PBS. Following the final wash step, the plate was read in a spectrophotometer at 485/538 (Ex/Em) to measure the number of bacteria bound to the fibronectin.

### Inhibition of B6 Microbial Community Binding to Fibronectin (Adhesion Inhibition Assay)

Colons from SPF C57BL.6J mice were excised from a freshly euthanized mouse between the ages of 8-12 weeks old and fileted open longitudinally. An inoculating loop was used to scrape the contents into a 1.5 mL Eppendorf containing 1 mL of PBS. Contents were centrifuged at 400×G for 5 minutes; the supernatant was strained using a 70 um cell strainer. The flow through was washed twice with PBS and once with TBS containing 10 mM CaCl2 at 10,000×G for 2 minutes. After the third wash the sample was resuspended in 500 uL TBS 10 mM CaCl2 and Cell-Brite 488 Membrane Stain and allowed to incubate for 30 minutes at 37 degrees C. to allow for fluorescent labeling of the bacteria. 200 ul of the OD 0.8 labeled culture was added to fresh Eppendorf tubes. The bacteria were treated with a dilution series of VHH F7 (0-10 uM) and incubated for 2 hours at 37 degrees C. Following incubation, the treated bacteria were carefully plated onto a fibronectin coated plate in triplicate, sealed and incubated at 37 degrees C. for 1 hour. After the 1-hour incubation, the bacteria were decanted from the plate and the plate was gently rinsed twice with 200 ul/well of PBS. Following the final wash step, the plate was read in a spectrophotometer at 485/538 (Ex/Em) to measure the number of bacteria bound to the fibronectin.

### Inhibition of IL1R1 Signaling Using HEK-Blue™ IL-1R Reporter Cells

HEK-Blue™ IL-1R Reporter Cells were grown to confluence in selection media according to the manufacturer's instructions. Selected HEK-Blue cells were rinsed twice with pre-warmed PBS and then detached using PBS for 2-3 minutes at 37 degrees C. The flask was tapped gently to loosen the cells and then rinsed with pre-warmed test media and resuspended to 280,000 cells/mL. To a 96-well flat bottom plate, 180 ul of the HEK-Blue cell suspension (~50,000 cells) was added and the plate was incubated at 37 degrees C. and 5% CO2 overnight. The following day, the reporter cells were treated with 10 uM VHH F7 for 2 hours and then followed by IL1-beta stimulus for an additional 2 hours. After stimulus, the Quanti-Blue solution was prepared according to the manufacturer's protocol. 180 ul of the Quanti-Blue solution was added to each well on a new 96-well flat bottom plate. 20 ul of each HEK-Blue induced supernatant was plated in triplicate and the plate was sealed and incubated at 37 degrees C. for 1 hour before determining SEAP levels by spectrophotometer at 620-655 nm.

### Antibody Binding to FimH

The FimH protein (2 μM) was used to coat wells of high-binding 96-well microtiter plates overnight (4° C.). Various concentrations of antibody were added to the coated wells and incubated for 1 hr at room temperature. Then antibody bound to the FimH protein was detected using an Anti-Avi Tag antibody at 450 nm (OD₄₅₀).

### Antibody Binding to IL1R1

The IL1R1 protein (1 μM) was used to coat wells of high-binding 96-well microtiter plates overnight (4° C.). Various concentrations of antibody were added to predefined wells of the high-binding 96-well microtiter plates and incubated for 1 hr at room temperature. Then, the antibody bound to the IL1R1 protein was detected by an Anti-Avi Tag antibody at 450 nm (OD₄₅₀).

What is claimed is:

1. An antibody that binds to target of HGMB1 (ToH1), the antibody comprising:
a) a heavy chain variable domain comprising complementary determining regions HCDR1, HCDR2, and HCDR3 and
b) and light chain variable domain comprising complementary determining regions LCDR1, LCDR2, and LCDR3, wherein
i. HCDR1, HCDR2, and HCDR3 comprise amino acid sequences having at least 80% sequence identity to SEQ ID NO: 11, SEQ ID NO: 12, and SEQ ID NO: 13, respectively, and LCDR1, LCDR2, and LCDR3 comprise amino acid sequences having at least 80% sequence identity to SEQ ID NO: 14, SEQ ID NO: 15, and SEQ ID NO: 16, respectively;
ii. HCDR1, HCDR2, and HCDR3 comprise amino acid sequences having at least 80% sequence identity to SEQ ID NO: 17, SEQ ID NO: 18, and SEQ ID NO: 19, respectively, and LCDR1, LCDR2, and LCDR3 comprise amino acid sequences having at least 80% sequence identity to SEQ ID NO: 20, SEQ ID NO: 21, and SEQ ID NO: 22, respectively; or
iii. HCDR1, HCDR2, and HCDR3 comprise amino acid sequences having at least 80% sequence identity to SEQ ID NO: 23, SEQ ID NO: 24, and SEQ ID NO: 25, respectively, and LCDR1, LCDR2, and LCDR3 comprise amino acid sequences having at least 80% sequence identity to SEQ ID NO: 26, SEQ ID NO: 27, and SEQ ID NO: 28, respectively.

2. The antibody of claim 1, wherein:
a) HCDR1, HCDR2, and HCDR3 comprise amino acid sequences having at least 90% sequence identity to SEQ ID NO: 11, SEQ ID NO: 12, and SEQ ID NO: 13, respectively, and LCDR1, LCDR2, and LCDR3 comprise amino acid sequences having at least 90% sequence identity to SEQ ID NO: 14, SEQ ID NO: 15, and SEQ ID NO: 16, respectively;
b) HCDR1, HCDR2, and HCDR3 comprise amino acid sequences having at least 90% sequence identity to SEQ ID NO: 17, SEQ ID NO: 18, and SEQ ID NO: 19, respectively, and LCDR1, LCDR2, and LCDR3 comprise amino acid sequences having at least 90% sequence identity to SEQ ID NO: 20, SEQ ID NO: 21, and SEQ ID NO: 22, respectively; or
c) HCDR1, HCDR2, and HCDR3 comprise amino acid sequences having at least 90% sequence identity to SEQ ID NO: 23, SEQ ID NO: 24, and SEQ ID NO: 25, respectively, and LCDR1, LCDR2, and LCDR3 comprise amino acid sequences having at least 90% sequence identity to SEQ ID NO: 26, SEQ ID NO: 27, and SEQ ID NO: 28, respectively.

3. The antibody of claim 1, wherein:
a) HCDR1, HCDR2, and HCDR3 comprise amino acid sequences having at least 95% sequence identity to SEQ ID NO: 11, SEQ ID NO: 12, and SEQ ID NO: 13, respectively, and LCDR1, LCDR2, and LCDR3 comprise amino acid sequences having at least 95% sequence identity to SEQ ID NO: 14, SEQ ID NO: 15, and SEQ ID NO: 16, respectively;
b) HCDR1, HCDR2, and HCDR3 comprise amino acid sequences having at least 95% sequence identity to SEQ ID NO: 17, SEQ ID NO: 18, and SEQ ID NO: 19, respectively, and LCDR1, LCDR2, and LCDR3 comprise amino acid sequences having at least 95% sequence identity to SEQ ID NO: 20, SEQ ID NO: 21, and SEQ ID NO: 22, respectively; or
c) HCDR1, HCDR2, and HCDR3 comprise amino acid sequences having at least 95% sequence identity to SEQ ID NO: 23, SEQ ID NO: 24, and SEQ ID NO: 25, respectively, and LCDR1, LCDR2, and LCDR3 comprise amino acid sequences having at least 95% sequence identity to SEQ ID NO: 26, SEQ ID NO: 27, and SEQ ID NO: 28, respectively.

4. The antibody of claim 1, wherein:
a) HCDR1, HCDR2, and HCDR3 comprise SEQ ID NO: 11, SEQ ID NO: 12, and SEQ ID NO: 13, respectively, and LCDR1, LCDR2, and LCDR3 comprise SEQ ID NO: 14, SEQ ID NO: 15, and SEQ ID NO: 16, respectively;
b) HCDR1, HCDR2, and HCDR3 comprise SEQ ID NO: 17, SEQ ID NO: 18, and SEQ ID NO: 19, respectively, and LCDR1, LCDR2, and LCDR3 comprise SEQ ID NO: 20, SEQ ID NO: 21, and SEQ ID NO: 22, respectively; or
c) HCDR1, HCDR2, and HCDR3 comprise SEQ ID NO: 23, SEQ ID NO: 24, and SEQ ID NO: 25, respectively, and LCDR1, LCDR2, and LCDR3 comprise SEQ ID NO: 26, SEQ ID NO: 27, and SEQ ID NO: 28, respectively.

5. The antibody of claim any one of claims 1-4, comprising:
a) a heavy chain variable domain comprising a sequence having at least 80% sequence identity to SEQ ID NO: 1, SEQ ID NO: 3, or SEQ ID NO: 5; and
b) a light chain variable domain comprising a sequence having at least 80% sequence identity to SEQ ID NO: 2, SEQ ID NO: 4, or SEQ ID NO: 6.

6. The antibody of claim 5, comprising:
a) a heavy chain variable domain comprising a sequence having at least 90% sequence identity to SEQ ID NO: 1, SEQ ID NO: 3, or SEQ ID NO: 5; and
b) a light chain variable domain comprising a sequence having at least 90% sequence identity to SEQ ID NO: 2, SEQ ID NO: 4, or SEQ ID NO: 6.

7. The antibody of claim 5, comprising:
a) a heavy chain variable domain comprising a sequence having at least 95% sequence identity to SEQ ID NO: 1, SEQ ID NO: 3, or SEQ ID NO: 5; and
b) a light chain variable domain comprising a sequence having at least 95% sequence identity to SEQ ID NO: 2, SEQ ID NO: 4, or SEQ ID NO: 6.

8. The antibody of claim 5, comprising:
a) a heavy chain variable domain comprising SEQ ID NO: 1 and a light chain variable domain comprising SEQ ID NO: 2;
b) a heavy chain variable domain comprising SEQ ID NO: 3 and a light chain variable domain comprising SEQ ID NO: 4; or
c) a heavy chain variable domain comprising SEQ ID NO: 5 and a light chain variable domain comprising SEQ ID NO: 6.

9. The antibody of any one of claims 1-8, wherein the heavy chain variable domain and the light chain variable domain are connected by a linker.

10. The antibody of claim 9, wherein the linker comprises a sequence having at least 80% identity to SEQ ID NO: 10.

11. The antibody of claim 9, wherein the linker comprises SEQ ID NO: 10.

12. The antibody of any one of claims 1-11, wherein the antibody is a single chain variable fragment (scFv).

13. An antibody that binds to target of HGMB1 (ToH1), wherein the antibody is a nanobody having at least 80% identity to SEQ ID NO: 29 or SEQ ID NO: 30.

14. The antibody of claim 13 having at least 90% identity to SEQ ID NO: 29 or SEQ ID NO: 30.

15. The antibody of claim 13 having at least 95% identity to SEQ ID NO: 29 or SEQ ID NO: 30.

16. The antibody of any one of claims 1-15 for use in a method of treating, preventing, or diagnosing microbial disease and/or chronic inflammatory disease in a subject.

17. The antibody of claim 16, wherein the microbial disease is a microbial infection.

18. The antibody of claim 17, wherein the microbial infection is a bacterial infection.

19. The antibody of claim 18, wherein the chronic inflammatory disease is inflammatory bowel disease.

20. A method of treating or preventing microbial disease and/or chronic inflammatory disease in a subject, the method comprising providing to the subject an antibody that binds to target of HGMB1 (ToH1).

21. The method of claim 20, wherein the antibody comprises the antibody of any one of claims 1-15.

22. The method of claim 20 or claim 21, wherein the microbial disease comprises a microbial infection.

23. The method of claim 22, wherein the microbial infection is a bacterial infection.

24. The method of claim 22, wherein the chronic inflammatory disease is inflammatory bowel disease.

25. A method of diagnosing microbial disease and/or chronic inflammatory disease in a subject, the method comprising determining levels of target of HGMB1 (ToH1) in a sample obtained from a subject, and determining that the subject has a microbial disease when the level of ToH1 in the sample are equal to or above a threshold value.

26. The method of claim 25, wherein determining levels of ToH1 in the sample comprises contacting a sample obtained from a subject with an antibody that binds to target of HGMB1 (ToH1), and detecting the antibody in the sample.

27. The method of claim 26, wherein the antibody comprises the antibody of any one of claims 1-15.

28. The method of claim 26 or claim 27, wherein the microbial disease comprises a microbial infection.

29. The method of claim 28, wherein the microbial infection is a bacterial infection.

30. The method of claim 28, wherein the chronic inflammatory disease is inflammatory bowel disease.

31. A method of generating an antibody that binds to target of HGMB1 (ToH1), the method comprising:
a) sequentially immunizing a host with two or more unique peptides, each of the two or more unique peptides having the motif [S/T]xExPx[I/V], wherein each x is a variable amino acid, and
b) isolating antibodies generated in response to immunization.

32. The method of claim 31, wherein the two or more unique peptides comprises three unique peptides.

33. The method of claim 32, wherein the two or more unique peptides comprises four unique peptides.

34. The method of claim 33, wherein the four unique peptides comprise SxExPxI, SxExPxV, TxExPxI, and TxExPxV.

35. The method of claim 34, wherein the four unique peptides comprise SAENPKI (SEQ ID NO: 42), SPEKPTV (SEQ ID NO: 36), TAEDPRI (SEQ ID NO: 41), and TSETPRV (SEQ ID NO: 33), or wherein the four unique peptides comprise TSETPRV (SEQ ID NO: 33), SSERPMI (SEQ ID NO: 34), SPEKPTV (SEQ ID NO: 36), and TRELPQI (SEQ ID NO: 37).

36. An antibody produced by the method of any one of claims 31-35.


SEQUENCE LISTING (61 sequences)

SEQ ID NO: 1 - protein, 118 aa, Synthetic construct
EVQLVQSGPEVKKPGASVKVSCKASGFIFSNYGIGWVRQAPGQGLEWLGWISGYNGQTNYAQTVQGRVTMTADTSTTTAYMDLRNLRSDDTAIYYCARQSIPYYMDVWGKGTMVTVSS

SEQ ID NO: 2 - protein, 112 aa, Synthetic construct
DIVMTQSPLSSPVTLGQPASISCRSSQSLVHSNGNTYLSWLHQRPGQPPRLLIYRISNRLSGVPDRFSGSGAGTDFTLKISRVEAEDVGVYYCMQAKQFPVTFGQGTRLEIK

SEQ ID NO: 3 - protein, 122 aa, Synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDRGSGATRYGMDVWGQGTLVTVSS

SEQ ID NO: 4 - protein, 110 aa, Synthetic construct
QSALTQPASVSGSPGQSITISCTGTSSDIGNYNYVSWYQQHPGKAPKLMIYDVTKRPSGVSNRLSGSKSGNTASLTISGLQAEDEADYYCSSYTGRSSWVFGGGTKLTVL

SEQ ID NO: 5 - protein, 121 aa, Synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARRSTWGTFDYWGQGTLVTVSS

SEQ ID NO: 6 - protein, 113 aa, Synthetic construct
DIVMTQTPDSLAVSLGERATINCKSSQSVLYSSNNKNYLAWYQQKPGQPPKLLIYWASTRESGVPDRFSGSGSGTDFTLTISSLQAEDVAVYYCQQYYALPLTFGGGTKLEIK

SEQ ID NO: 7 - protein, 281 aa, Synthetic construct
MGYLLPTAAAGLLLLAAQPAMAEVQLVQSGPEVKKPGASVKVSCKASGFIFSNYGIGWVRQAPGQGLEWLGWISGYNGQTNYAQTVQGRVTMTADTSTTTAYMDLRNLRSDDTAIYYCARQSIPYYMDVWGKGTMVTVSSGGGGSGGGGSGGGGSGGGGASDIVMTQSPLSSPVTLGQPASISCRSSQSLVHSNGNTYLSWLHQRPGQPPRLLIYRISNRLSGVPDRFSGSGAGTDFTLKISRVEAEDVGVYYCMQAKQFPVTFGQGTRLEIKGSHHHHHH

SEQ ID NO: 8 - protein, 283 aa, Synthetic construct
MGYLLPTAAAGLLLLAAQPAMAQVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDRGSGATRYGMDVWGQGTLVTVSSGGGGSGGGGSGGGGSGGGGASQSALTQPASVSGSPGQSITISCTGTSSDIGNYNYVSWYQQHPGKAPKLMIYDVTKRPSGVSNRLSGSKSGNTASLTISGLQAEDEADYYCSSYTGRSSWVFGGGTKLTVLGSHHHHHH

SEQ ID NO: 9 - protein, 285 aa, Synthetic construct
MGYLLPTAAAGLLLLAAQPAMAQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARRSTWGTFDYWGQGTLVTVSSGGGGSGGGGSGGGGSGGGGASDIVMTQTPDSLAVSLGERATINCKSSQSVLYSSNNKNYLAWYQQKPGQPPKLLIYWASTRESGVPDRFSGSGSGTDFTLTISSLQAEDVAVYYCQQYYALPLTFGGGTKLEIKGSHHHHHH

SEQ ID NO: 10 - protein, 21 aa, Synthetic construct
GGGGSGGGGSGGGGSGGGGAS

SEQ ID NO: 11 - protein, 8 aa, Synthetic construct
GFIFSNYG

SEQ ID NO: 12 - protein, 8 aa, Synthetic construct
ISGYNGQT

SEQ ID NO: 13 - protein, 11 aa, Synthetic construct
ARQSIPYYMDV

SEQ ID NO: 14 - protein, 11 aa, Synthetic construct
QSLVHSNGNTY

SEQ ID NO: 15 - protein, 14 aa, Synthetic construct
RISNRLSGVPDRFS

SEQ ID NO: 16 - protein, 9 aa, Synthetic construct
MQAKQFPVT

SEQ ID NO: 17 - protein, 8 aa, Synthetic construct
GYTFTGYY

SEQ ID NO: 18 - protein, 8 aa, Synthetic construct
INPNSGGT

SEQ ID NO: 19 - protein, 15 aa, Synthetic construct
ARDRGSGATRYGMDV

SEQ ID NO: 20 - protein, 9 aa, Synthetic construct
SSDIGNYNY

SEQ ID NO: 21 - protein, 21 aa, Synthetic construct
DVTKRPSGVSNRLSGSKSGNT

SEQ ID NO: 22 - protein, 10 aa, Synthetic construct
SSYTGRSSWV

SEQ ID NO: 23 - protein, 10 aa, Synthetic construct
GDSVSSNSAA

SEQ ID NO: 24 - protein, 9 aa, Synthetic construct
TYYRSKWYN

SEQ ID NO: 25 - protein, 11 aa, Synthetic construct
ARRSTWGTFDY

SEQ ID NO: 26 - protein, 12 aa, Synthetic construct
QSVLYSSNNKNY

SEQ ID NO: 27 - protein, 14 aa, Synthetic construct
WASTRESGVPDRFS

SEQ ID NO: 28 - protein, 9 aa, Synthetic construct
QQYYALPLT

SEQ ID NO: 29 - protein, 122 aa, Synthetic construct
QVQLVESGGGLVQPGGSLRLSCAASGFTMGDYAIGWFRQVPGKEREGLSYSTKNGIKAYADSVRDRFTISRENAKNTVYLQMNSLKPEDTAVYYCAASTRFGGLLVSRYDYWGQGTQVTVSS

SEQ ID NO: 30 - protein, 122 aa, Synthetic construct
QLQLVESGGGLVQAGGSLRLSCAASGLTVSSYSMGWFRQAPGKEREFVAAISRSGATINYGSSVQGRFMIARDDAKNTVNLQMNSLKPEDTAVYYCAARDRYSLVARAYEYWGQGTQVTVSS

SEQ ID NO: 31 - protein, 164 aa, Synthetic construct
MKHLWFFLLLVAAPRWVLSQVQLVESGGGLVQPGGSLRLSCAASGFTMGDYAIGWFRQVPGKEREGLSYSTKNGIKAYADSVRDRFTISRENAKNTVYLQMNSLKPEDTAVYYCAASTRFGGLLVSRYDYWGQGTQVTVSSGLNDIFEAQKIEWHEGSHHHHHH

SEQ ID NO: 32 - protein, 107 aa, Synthetic construct
QAPGKEREFVAAISRSGATINYGSSVQGRFMIARDDAKNTVNLQMNSLKPEDTAVYYCAARDRYSLVARAYEYWGQGTQVTVSSGLNDIFEAQKIEWHEGSHHHHHH

SEQ ID NO: 33 - protein, 7 aa, Synthetic construct
TSETPRV

SEQ ID NO: 34 - protein, 7 aa, Synthetic construct
SSERPMI

SEQ ID NO: 35 - protein, 7 aa, Synthetic construct
SFELPTI

SEQ ID NO: 36 - protein, 7 aa, Synthetic construct
SPEKPTV

SEQ ID NO: 37 - protein, 7 aa, Synthetic construct
TRELPQI

SEQ ID NO: 38 - protein, 7 aa, Synthetic construct
TFEPPVV

SEQ ID NO: 39 - protein, 7 aa, Synthetic construct
TKENPFV

SEQ ID NO: 40 - protein, 7 aa, Synthetic construct
SYERPDI

SEQ ID NO: 41 - protein, 7 aa, Synthetic construct
TAEDPRI

SEQ ID NO: 42 - protein, 7 aa, Synthetic construct
SAENPKI

SEQ ID NO: 43 - DNA, 18 bp, Synthetic construct
  source 1..18: mol_type = other DNA
gctgcctcccgtaggagt

SEQ ID NO: 44 - DNA, 20 bp, Synthetic construct
  source 1..20: mol_type = other DNA
ggcattcgggctcctttctt

SEQ ID NO: 45 - DNA, 22 bp, Synthetic construct
  source 1..22: mol_type = other DNA
tggagtggtagtcgatgctaag

SEQ ID NO: 46 - DNA, 20 bp, Synthetic construct
  source 1..20: mol_type = other DNA
agggctcggaactccagaaa

SEQ ID NO: 47 - DNA, 21 bp, Synthetic construct
  source 1..21: mol_type = other DNA
ccagggaatcggtagacatcg

SEQ ID NO: 48 - DNA, 21 bp, Synthetic construct
  source 1..21: mol_type = other DNA
gccgtgaattgtatgaacgga

SEQ ID NO: 49 - DNA, 21 bp, Synthetic construct
  source 1..21: mol_type = other DNA
cgcagttgaccacgttgacta

SEQ ID NO: 50 - DNA, 21 bp, Synthetic construct
  source 1..21: mol_type = other DNA
aggtcggtgtgaacggatttg

SEQ ID NO: 51 - DNA, 23 bp, Synthetic construct
  source 1..23: mol_type = other DNA
tgtagaccatgtagttgaggtca

SEQ ID NO: 52 - DNA, 20 bp, Synthetic construct
  source 1..20: mol_type = other DNA
actcctacgggaggcagcag

SEQ ID NO: 53 - DNA, 17 bp, Synthetic construct
  source 1..17: mol_type = other DNA
attaccgcggctgctgg

SEQ ID NO: 54 - DNA, 21 bp, Synthetic construct
  source 1..21: mol_type = other DNA
gctggtggtaggaaatggata

SEQ ID NO: 55 - DNA, 20 bp, Synthetic construct
  source 1..20: mol_type = other DNA
cttatggcggcgtgttatct

SEQ ID NO: 56 - DNA, 20 bp, Synthetic construct
  source 1..20: mol_type = other DNA
gagcaagcggacctcataaa

SEQ ID NO: 57 - DNA, 23 bp, Synthetic construct
  source 1..23: mol_type = other DNA
ggcattctgatccacgattacta

SEQ ID NO: 58 - DNA, 39 bp, Synthetic construct
  source 1..39: mol_type = other DNA
ggccgggatccaaacgagttattaccctgtttgctgtac

SEQ ID NO: 59 - DNA, 42 bp, Synthetic construct
  source 1..42: mol_type = other DNA
gcgcgctcgagttattgataaacaaaagtcacgccaataatc

SEQ ID NO: 60 - DNA, 45 bp, Synthetic construct
  source 1..45: mol_type = other DNA
agcgcgggccgtagcgcttgcggtaggaaatggatagctactgcc

SEQ ID NO: 61 - DNA, 48 bp, Synthetic construct
  source 1..48: mol_type = other DNA
gcaagcgctacggcccgcgctgtttataattcgagaacggataagccg